SEQUENCE LISTING (18 sequences)

SEQ ID NO: 1 - protein, 317 aa, Homo sapien
MRRASRDYTKYLRGSEEMGGGPGAPHEGPLHAPPPPAPHQPPAASRSMFVALLGLGLGQVVCSVALFFYFRAQMDPNRISEDGTHCIYRILRLHENADFQDTTLESQDTKLIPDSCRRIKQAFQGAVQKELQHIVGSQHIRAEKAMVDGSWLDLAKRSKLEAQPFAHLTINATDIPSGSHKVSLSSWYHDRGWAKISNMTFSNGKLIVNQDGFYYLYANICFRHHETSGDLATEYLQLMVYVTKTSIKIPSSHTLMKGGSTKYWSGNSEFHFYSINVGGFFKLRSGEEISIEVSNPSLLDPDQDATYFGAFKVRDID

SEQ ID NO: 2 - DNA, 2271 bp, Homo sapien
aagcttggtaccgagctcggatccactactcgacccacgcgtccgcgcgccccaggagccaaagccgggctccaagtcggcgccccacgtcgaggctccgccgcagcctccggagttggccgcagacaagaaggggagggagcgggagagggaggagagctccgaagcgagagggccgagcgccatgcgccgcgccagcagagactacaccaagtacctgcgtggctcggaggagatgggcggcggccccggagccccgcacgagggccccctgcacgccccgccgccgcctgcgccgcaccagccccccgccgcctcccgctccatgttcgtggccctcctggggctggggctgggccaggttgtctgcagcgtcgccctgttcttctatttcagagcgcagatggatcctaatagaatatcagaagatggcactcactgcatttatagaattttgagactccatgaaaatgcagattttcaagacacaactctggagagtcaagatacaaaattaatacctgattcatgtaggagaattaaacaggcctttcaaggagctgtgcaaaaggaattacaacatatcgttggatcacagcacatcagagcagagaaagcgatggtggatggctcatggttagatctggccaagaggagcaagcttgaagctcagccttttgctcatctcactattaatgccaccgacatcccatctggttcccataaagtgagtctgtcctcttggtaccatgatcggggttgggccaagatctccaacatgacttttagcaatggaaaactaatagttaatcaggatggcttttattacctgtatgccaacatttgctttcgacatcatgaaacttcaggagacctagctacagagtatcttcaactaatggtgtacgtcactaaaaccagcatcaaaatcccaagttctcataccctgatgaaaggaggaagcaccaagtattggtcagggaattctgaattccatttttattccataaacgttggtggattttttaagttacggtctggagaggaaatcagcatcgaggtctccaacccctccttactggatccggatcaggatgcaacatactttggggcttttaaagttcgagatatagattgagccccagtttttggagtgttatgtatttcctggatgtttggaaacattttttaaaacaagccaagaaagatgtatataggtgtgtgagactactaagaggcatggccccaacggtacacgactcagtatccatgctcttgaccttgtagagaacacgcgtatttacagccagtgggagatgttagactcatggtgtgttacacaatggtttttaaattttgtaatgaattcctagaattaaaccagattggagcaattacgggttgaccttatgagaaactgcatgtgggctatgggaggggttggtccctggtcatgtgccccttcgcagctgaagtggagagggtgtcatctagcgcaattgaaggatcatctgaaggggcaaattcttttgaattgttacatcatgctggaacctgcaaaaaatactttttctaatgaggagagaaaatatatgtatttttatataatatctaaagttatatttcagatgtaatgttttctttgcaaagtattgtaaattatatttgtgctatagtatttgattcaaaatatttaaaaatgtcttgctgttgacatatttaatgttttaaatgtacagacatatttaactggtgcactttgtaaattccctggggaaaacttgcagctaaggaggggaaaaaaatgttgtttcctaatatcaaatgcagtatatttcttcgttctttttaagttaatagattttttcagacttgtcaagcctgtgcaaaaaaattaaaatggatgccttgaataataagcaggatgttggccaccaggtgcctttcaaatttagaaactaattgactttagaaagctgacattgccaaaaaggatacataatgggccactgaaatctgtcaagagtagttatataattgttgaacaggtgtttttccacaagtgccgcaaattgtacctttttttttttttcaaaatagaaaagttattagtggtttatcagcaaaaaagtccaattttaatttagtaaatgttatcttatactgtacaataaaaacattgcctttgaatgttaattttttggtacaaaaataaatttatatgaaaaaaaaaaaaaaagggcggccgctctagagggccctattctatag

SEQ ID NO: 3 - protein, 401 aa, Homo sapien
MNKLLCCALVFLDISIKWTTQETFPPKYLHYDEETSHQLLCDKCPPGTYLKQHCTAKWKTVCAPCPDHYYTDSWHTSDECLYCSPVCKELQYVKQECNRTHNRVCECKEGRYLEIEFCLKHRSCPPGFGVVQAGTPERNTVCKRCPDGFFSNETSSKAPCRKHTNCSVFGLLLTQKGNATHDNICSGNSESTQKCGIDVTLCEEAFFRFAVPTKFTPNWLSVLVDNLPGTKVNAESVERIKRQHSSQEQTFQLLKLWKHQNKAQDIVKKIIQDIDLCENSVQRHIGHANLTFEQLRSLMESLPGKKVGAEDIEKTIKACKPSDQILKLLSLWRIKNGDQDTLKGLMHALKHSKTYHFPKTVTQSLKKTIRFLHSFTMYKLYQKLFLEMIGNQVQSVKISCL

SEQ ID NO: 4 - DNA, 1356 bp, Homo sapien
gtatatataacgtgatgagcgtacgggtgcggagacgcaccggagcgctcgcccagccgccgyctccaagcccctgaggtttccggggaccacaatgaacaagttgctgtgctgcgcgctcgtgtttctggacatctccattaagtggaccacccaggaaacgtttcctccaaagtaccttcattatgacgaagaaacctctcatcagctgttgtgtgacaaatgtcctcctggtacctacctaaaacaacactgtacagcaaagtggaagaccgtgtgcgccccttgccctgaccactactacacagacagctggcacaccagtgacgagtgtctatactgcagccccgtgtgcaaggagctgcagtacgtcaagcaggagtgcaatcgcacccacaaccgcgtgtgcgaatgcaaggaagggcgctaccttgagatagagttctgcttgaaacataggagctgccctcctggatttggagtggtgcaagctggaaccccagagcgaaatacagtttgcaaaagatgtccagatgggttcttctcaaatgagacgtcatctaaagcaccctgtagaaaacacacaaattgcagtgtctttggtctcctgctaactcagaaaggaaatgcaacacacgacaacatatgttccggaaacagtgaatcaactcaaaaatgtggaatagatgttaccctgtgtgaggaggcattcttcaggtttgctgttcctacaaagtttacgcctaactggcttagtgtcttggtagacaatttgcctggcaccaaagtaaacgcagagagtgtagagaggataaaacggcaacacagctcacaagaacagactttccagctgctgaagttatggaaacatcaaaacaaagcccaagatatagtcaagaagatcatccaagatattgacctctgtgaaaacagcgtgcagcggcacattggacatgctaacctcaccttcgagcagcttcgtagcttgatggaaagcttaccgggaaagaaagtgggagcagaagacattgaaaaaacaataaaggcatgcaaacccagtgaccagatcctgaagctgctcagtttgtggcgaataaaaaatggcgaccaagacaccttgaagggcctaatgcacgcactaaagcactcaaagacgtaccactttcccaaaactgtcactcagagtctaaagaagaccatcaggttccttcacagcttcacaatgtacaaattgtatcagaagttatttttagaaatgataggtaaccaggtccaatcagtaaaaataagctgcttataactggaaatggccattgagctgtttcctcacaattggcgagatcccatggatgataa

SEQ ID NO: 5 - protein, 616 aa, Homo sapien
MAPRARRRRPLFALLLLCALLARLQVALQIAPPCTSEKHYEHLGRCCNKCEPGKYMSSKCTTTSDSVCLPCGPDEYLDSWNEEDKCLLHKVCDTGKALVAVVAGNSTTPRRCACTAGYHWSQDCECCRRNTECAPGLGAQHPLQLNKDTVCKPCLAGYFSDAFSSTDKCRPWTNCTFLGKRVEHHGTEKSDAVCSSSLPARKPPNEPHVYLPGLIILLLFASVALVAAIIFGVCYRKKGKALTANLWHWINEACGRLSGDKESSGDSCVSTHTANFGQQGACEGVLLLTLEEKTFPEDMCYPDQGGVCQGTCVGGGPYAQGEDARMLSLVSKTEIEEDSFRQMPTEDEYMDRPSQPTDQLLFLTEPGSKSTPPFSEPLEVGENDSLSQCFTGTQSTVGSESCNCTEPLCRTDWTPMSSENYLQKEVDSGHCPHWAASPSPNWADVCTGCRNPPGEDCEPLVGSPKRGPLPQCAYGMGLPPEEEASRTEARDQPEDGADGRLPSSARAGAGSGSSPGGQSPASGNVTGNSNSTFISSGQVMNFKGDIIVVYVSQTSQEGAAAAAEPMGRPVQEETLARRDSFAGNGPRFPDPCGGPEGLREPEKASRPVQEQGGAKA

SEQ ID NO: 6 - DNA, 3136 bp, Homo sapien
ccgctgaggccgcggcgcccgccagcctgtcccgcgccatggccccgcgcgcccggcggcgccgcccgctgttcgcgctgctgctgctctgcgcgctgctcgcccggctgcaggtggctttgcagatcgctcctccatgtaccagtgagaagcattatgagcatctgggacggtgctgtaacaaatgtgaaccaggaaagtacatgtcttctaaatgcactactacctctgacagtgtatgtctgccctgtggcccggatgaatacttggatagctggaatgaagaagataaatgcttgctgcataaagtttgtgatacaggcaaggccctggtggccgtggtcgccggcaacagcacgaccccccggcgctgcgcgtgcacggctgggtaccactggagccaggactgcgagtgctgccgccgcaacaccgagtgcgcgccgggcctgggcgcccagcacccgttgcagctcaacaaggacacagtgtgcaaaccttgccttgcaggctacttctctgatgccttttcctccacggacaaatgcagaccctggaccaactgtaccttccttggaaagagagtagaacatcatgggacagagaaatccgatgcggtttgcagttcttctctgccagctagaaaaccaccaaatgaaccccatgtttacttgcccggtttaataattctgcttctcttcgcgtctgtggccctggtggctgccatcatctttggcgtttgctataggaaaaaagggaaagcactcacagctaatttgtggcactggatcaatgaggcttgtggccgcctaagtggagataaggagtcctcaggtgacagttgtgtcagtacacacacggcaaactttggtcagcagggagcatgtgaaggtgtcttactgctgactctggaggagaagacatttccagaagatatgtgctacccagatcaaggtggtgtctgtcagggcacgtgtgtaggaggtggtccctacgcacaaggcgaagatgccaggatgctctcattggtcagcaagaccgagatagaggaagacagcttcagacagatgcccacagaagatgaatacatggacaggccctcccagcccacagaccagttactgttcctcactgagcctggaagcaaatccacacctcctttctctgaacccctggaggtgggggagaatgacagtttaagccagtgcttcacggggacacagagcacagtgggttcagaaagctgcaactgcactgagcccctgtgcaggactgattggactcccatgtcctctgaaaactacttgcaaaaagaggtggacagtggccattgcccgcactgggcagccagccccagccccaactgggcagatgtctgcacaggctgccggaaccctcctggggaggactgtgaacccctcgtgggttccccaaaacgtggacccttgccccagtgcgcctatggcatgggccttccccctgaagaagaagccagcaggacggaggccagagaccagcccgaggatggggctgatgggaggctcccaagctcagcgagggcaggtgccgggtctggaagctcccctggtggccagtcccctgcatctggaaatgtgactggaaacagtaactccacgttcatctccagcgggcaggtgatgaacttcaagggcgacatcatcgtggtctacgtcagccagacctcgcaggagggcgcggcggcggctgcggagcccatgggccgcccggtgcaggaggagaccctggcgcgccgagactccttcgcggggaacggcccgcgcttcccggacccgtgcggcggccccgaggggctgcgggagccggagaaggcctcgaggccggtgcaggagcaaggcggggccaaggcttgagcgccccccatggctgggagcccgaagctcggagccagggctcgcgagggcagcaccgcagcctctgccccagccccggccacccagggatcgatcggtacagtcgaggaagaccacccggcattctctgcccactttgccttccaggaaatgggcttttcaggaagtgaattgatgaggactgtccccatgcccacggatgctcagcagcccgccgcactggggcagatgtctcccctgccactcctcaaactcgcagcagtaatttgtggcactatgacagctatttttatgactatcctgttctgtggggggggggtctatgttttccccccatatttgtattccttttcataacttttcttgatatctttcctccctcttttttaatgtaaaggttttctcaaaaattctcctaaaggtgagggtctctttcttttctcttttccttttttttttctttttttggcaacctggctctggcccaggctagagtgcagtggtgcgattatagcccggtgcagcctctaactcctgggctcaagcaatccaagtgatcctcccacctcaaccttcggagtagctgggatcacagctgcaggccacgcccagcttcctccccccgactccccccccccagagacacggtcccaccatgttacccagcctggtctcaaactccccagctaaagcagtcctccagcctcggcctcccaaagtactgggattacaggcgtgagcccccacgctggcctgctttacgtattttcttttgtgcccctgctcacagtgttttagagatggctttcccagtgtgtgttcattgtaaacacttttgggaaagggctaaacatgtgaggcctggagatagttgctaagttgctaggaacatgtggtgggactttcatattctgaaaaatgttctatattctcatttttctaaaagaaagaaaaaaggaaacccgatttatttctcctgaatctttttaagtttgtgtcgttccttaagcagaactaagctcagtatgtgaccttacccgctaggtggttaatttatccatgctggcagaggcactcaggtacttggtaagcaaatttctaaaactccaagttgctgcagcttggcattcttcttattctagaggtctctctggaaaagatggagaaaatgaacaggacatggggctcctggaaagaaagggcccgggaagttcaaggaagaataaagttgaaattttaaaaaaaaa

SEQ ID NO: 7 - DNA, 20 bp, Artificial Sequence
  sequence is synthesized
agtggtgcgattatagcccg

SEQ ID NO: 8 - DNA, 21 bp, Artificial Sequence
  sequence is synthesized
gaaggttgaggtgggaggatc

SEQ ID NO: 9 - DNA, 28 bp, Artificial Sequence
  sequence is synthesized
agcctctaactcctgggctcaagcaatc

SEQ ID NO: 10 - DNA, 20 bp, Artificial Sequence
  sequence is synthesized
tgggctacactgagcaccag

SEQ ID NO: 11 - DNA, 20 bp, Artificial Sequence
  sequence is synthesized
cagcgtcaaaggtggaggag

SEQ ID NO: 12 - DNA, 28 bp, Artificial Sequence
  sequence is synthesized
tggtctcctctgacttcaacagcgacac

SEQ ID NO: 13 - DNA, 23 bp, Artificial Sequence
  sequence is synthesized
caagtattggtcagggaattctg

SEQ ID NO: 14 - DNA, 24 bp, Artificial Sequence
  sequence is synthesized
gggctcaatctatatctcgaactt

SEQ ID NO: 15 - DNA, 31 bp, Artificial Sequence
  sequence is synthesized
tttaagttacggtctggagaggaaatcagca

SEQ ID NO: 16 - DNA, 19 bp, Artificial Sequence
  sequence is synthesized
gaaggtgaaggtcggagtc

SEQ ID NO: 17 - DNA, 20 bp, Artificial Sequence
  sequence is synthesized
gaagatggtgatgggatttc

SEQ ID NO: 18 - DNA, 20 bp, Artificial Sequence
  sequence is synthesized
caagcttcccgttctcagcc

(19) United States
(12) Patent Application Publication
(10) Pub. No.: US 2005/0031583 A1
(43) Pub. Date: Feb. 10, 2005

(54) Uses of opg to modulate immune responses

(76) Inventor: Iqbal Grewal, Fremont, CA (US)

Correspondence Address: GENENTECH, INC., 1 DNA WAY, SOUTH SAN FRANCISCO, CA, 94080, US

(21) Appl. No.: 10/129,595

(22) PCT Filed: Feb. 6, 2002
(86) PCT No.: PCT/US02/01238

Related U.S. Application Data
(60) Provisional application No. 60/278,215, filed on Mar. 23, 2001.

(51) Int. Cl. A61K 38/20; C12N 5/08

(52) U.S. Cl. 424/85.2; 435/372

(57) ABSTRACT

Methods of stimulating or inhibiting activity of monocytes using OPG ligand, or other agonists or antagonists, are provided. Methods of treating pathological conditions, particularly immune related conditions, using such OPG ligand, agonists or antagonists are further provided. Agonists and antagonists contemplated for use in the invention include anti-RANK receptor antibodies, anti-OPG ligand antibodies, anti-OPG receptor antibodies, RANK receptor immunoadhesins, and OPG receptor immunoadhesins.

### FIELD OF THE INVENTION

This invention relates generally to methods of using the tumor necrosis factor (TNF) family-related molecule, OPG Ligand, or other agonists or antagonists, to modulate immune system activity.

### BACKGROUND OF THE INVENTION

Various molecules, such as tumor necrosis factor-α (“TNF-α”), tumor necrosis factor-β (“TNF-β” or “lymphotoxin-α”), lymphotoxin-β (“LT-β”), CD30 ligand, CD27 ligand, CD40 ligand, OX-40 ligand, 4-1BB ligand, Apo-1 ligand (also referred to as Fas ligand or CD95 ligand), Apo-2 ligand (also referred to as TRAIL), Apo-3 ligand (also referred to as TWEAK), APRIL, OPG ligand (also referred to as RANK ligand, ODF, or TRANCE), and TALL-1 (also referred to as BlyS, BAFF or THANK) have been identified as members of the tumor necrosis factor (“TNF”) family of cytokines (See, e.g., Gruss and Dower, Blood, 85:3378-3404 (1995); Pitti et al., J. Biol. Chem., 271:12687-12690 (1996); Wiley et al., Immunity, 3:673-682 (1995); Browning et al., Cell, 72:847-856 (1993); Armitage et al. Nature, 357:80-82 (1992), WO 97/01633 published Jan. 16, 1997; WO 97/25428 published Jul. 17, 1997; Marsters et al., Curr. Biol., 8:525-528 (1998); Chicheportiche et al., Biol. Chem., 272:32401-32410 (1997); Hahne et al., J. Exp. Med., 188:1185-1190 (1998); WO98/28426 published Jul. 2, 1998; WO98/46751 published Oct. 22, 1998; WO/98/18921 published May 7, 1998; Moore et al., Science, 285:260-263 (1999); Shu et al., J. Leukocyte Biol., 65:680 (1999); Schneider et al., J. Exp. Med., 189:1747-1756 (1999); Mukhopadhyay et al., J. Biol. Chem., 274:15978-15981 (1999)]. Among these molecules, TNF-α, TNF-β, CD30 ligand, 4-1BB ligand, Apo-1 ligand, Apo-2 ligand (Apo2L/TRAIL) and Apo-3 ligand (TWEAK) have been reported to be involved in apoptotic cell death. Both TNF-α and TNF-β have been reported to induce apoptotic death in susceptible tumor cells [Schmid et al., Proc. Natl. Acad. Sci., 83:1881 (1986); Dealtry et al., Eur. J. Immunol., 17:689 (1987)].

Various molecules in the TNF family also have purported role(s) in the function or development of the immune system [Gruss et al., Blood, 85:3378 (1995)). Zheng et al. have reported that TNF-α is involved in post-stimulation apoptosis of CD8-positive T cells [Zheng et al., Nature, 377:348-351 (1995)]. Other investigators have reported that CD30 ligand may be involved in deletion of self-reactive T cells in the thymus [Amakawa et al., Cold Spring Harbor Laboratory Symposium on Programmed Cell Death, Abstr. No. 10, (1995)]. CD40 ligand activates many functions of B cells, including proliferation, immunoglobulin secretion, and survival (Renshaw et al., J. Exp. Med., 180:1889 (1994)]. Another recently identified TNF family cytokine, TALL-1 (BlyS), has been reported, under certain conditions, to induce B cell proliferation and immunoglobulin secretion. [Moore et al., supra; Schneider et al., supra; Mackay et al., J. Exp. Med., 190:1697 (1999)].

Mutations in the mouse Fas/Apo-1 receptor or ligand genes (called 1pr and gld, respectively) have been associated with some autoimmune disorders, indicating that Apo-1 ligand may play a role in regulating the clonal deletion of self-reactive lymphocytes in the periphery [Krammer et al., Curr. Op. Immunol., 6:279-289 (1994); Nagata et al., Science, 267:1449-1456 (1995)]. Apo-1 ligand is also reported to induce post-stimulation apoptosis in CD4-positive T lymphocytes and in B lymphocytes, and may be involved in the elimination of activated lymphocytes when their function is no longer needed [Krammer et al., supra; Nagata et al., supra]. Agonist mouse monoclonal antibodies specifically binding to the Apo-1 receptor have been reported to exhibit cell killing activity that is comparable to or similar to that of TNF-α [Yonehara et al., J. Exp. Med., 169:1747-1756 (1989)].

The TNF-related ligand called OPG ligand (also referred to as RANK ligand, TRANCE, or ODF) has been reported in the literature to have some involvement in certain immunoregulatory activities. WO98/28426 published Jul. 2, 1998 describes the ligand (referred to therein as RANK ligand) as a Type 2 transmembrane protein, which in a soluble form, was found to induce maturation of dendritic cells, enhance CD 1a+ dendritic cell allo-stimulatory capacity in a MLR, and enhance the number of viable human peripheral blood T cells in vitro in the presence of TGF-beta. [see also, Anderson et al., Nature, 390:175-179 (1997); WO 99/29865 published Jun. 17, 1999]. The WO98/28426 reference also discloses that the ligand enhanced production of TNF-alpha by one macrophage tumor cell line (called RAW264.7; ATCC TIB71), but did not stimulate nitric oxide production by those tumor cells. [See, also, Nagai et al., Biochem. Biophys. Res. Comm., 269:532-536 (2000); WO 00/15807 published Mar. 23, 2000].

The putative roles of OPG ligand/TRANCE/ODF in modulating dendritic cell activity [see, e.g., Wong et al., J. Exp. Med., 186:2075-2080 (1997); Wong et al., J. Leukocyte Biol., 65:715-724 (1999); Wong et al., J. Biol. Chem., 272:25190-25194 (1997); Josien et al., J. Immunol., 162:2562-2568 (1999); Josien et al., J. Exp. Med., 191495-501 (2000)] and in influencing T cell activation in an immune response [see, e.g., Bachmann et al., J. Exp. Med., 189:1025-1031 (1999); Green et al., J. Exp. Med., 189:1017-1020 (1999)] have been explored in the literature. Kong et al., Nature, 397:315-323 (1999) report that mice with a disrupted opgl gene showed severe osteoporosis, lacked osteoclasts, and exhibited defects in early differentiation of T and B lymphocytes. Kong et al. have further reported that systemic activation of T cells in vivo led to an OPGL-mediated increase in osteoclastogenesis and bone loss. [Kong et al., Nature, 402:304-308 (1999)].

The TNFR family member, referred to as RANK, has been identified as a receptor for OPG ligand (see WO98/28426 published Jul. 2, 1998; WO 99/58674 published Nov. 18, 1999; Anderson et al., Nature, 390:175-179 (1997); Lacey et al., Cell, 93:165-176 (1998). Another TNFR-related molecule, called OPG (FDCR-1 or OCIF), has also been identified as a receptor for OPG ligand. [Simonet et al., Cell, 89:309 (1997); Yasuda et al., Endocrinology, 139:1329 (1998); Yun et al., J. Immunol., 161:6113-6121 (1998)]. Yun et al., supra, disclose that OPG/FDCR-1/OCIF is expressed in both a membrane-bound form and a secreted form and has a restricted expression pattern in cells of the immune system, including dendritic cells, EBV-transformed B cell lines and tonsillar B cells. Yun et al. also disclose that in B cells and dendritic cells, expression of OPG/FDCR-1/OCIF can be up-regulated by CD40, a molecule involved in B cell activation. However, Yun et al. acknowledge that how OPG/FDCR-1/OCIF functions in the regulation of the immune response is unknown.

Induction of various cellular responses mediated by such TNF family cytokines is believed to be initiated by their binding to specific cell receptors. Previously, two distinct TNF receptors of approximately 55-kDa (TNFR1) and 75-kDa (TNFR2) were identified [Hohman et al., J. Biol. Chem., 264:14927-14934 (1989); Brockhaus et al., Proc. Natl. Acad. Sci., 87:3127-3131 (1990); EP 417,563, published Mar. 20, 1991; Loetscher et al., Cell, 61:351 (1990); Schall et al., Cell, 61:361 (1990); Smith et al., Science, 248:1019-1023 (1990); Lewis et al., Proc. Natl. Acad. Sci., 88:2830-2834 (1991); Goodwin et al., Mol. Cell. Biol., 11:3020-3026 (1991)]. Those TNFRs were found to share the typical structure of cell surface receptors including extracellular, transmembrane and intracellular regions. The extracellular portions of both receptors were found naturally also as soluble TNF-binding proteins [Nophar, Y. et al., EMBO J., 9:3269 (1990); and Kohno, T. et al., Proc. Natl. Acad. Sci. U.S.A., 87:8331 (1990); Hale et al., J. Cell. Biochem. Supplement 15F, 1991, p. 113 (P424)].

The extracellular portion of type 1 and type 2 TNFRs (TNFR1 and TNFR2) contains a repetitive amino acid sequence pattern of four cysteine-rich domains (CRDs) designated 1 through 4, starting from the NH₂-terminus. [Schall et al., supra; Loetscher et al., supra; Smith et al., supra; Nophar et al., supra; Kohno et al., supra; Banner et al., Cell, 73:431-435 (1993)]. A similar repetitive pattern of CRDs exists in several other cell-surface proteins, including the p75 nerve growth factor receptor (NGFR) [Johnson et al., Cell, 47:545 (1986); Radeke et al., Nature, 325:593 (1987)], the B cell antigen CD40 [Stamenkovic et al., EMBO J., 8:1403 (1989)], the T cell antigen OX40 [Mallet et al., EMBO J., 9:1063 (1990)] and the Fas antigen [Yonehara et al., supra and Itoh et al., Cell, 66:233-243 (1991)]. CRDs are also found in the soluble TNFR (sTNFR)-like T2 proteins of the Shope and myxoma poxviruses [Upton et al., Virology, 160:20-29 (1987); Smith et al., Biochem. Biophys. Res. Commun., 176:335 (1991); Upton et al., Virology, 184:370 (1991)]. Optimal alignment of these sequences indicates that the positions of the cysteine residues are well conserved. These receptors are sometimes collectively referred to as members of the TNF/NGF receptor superfamily.

The TNF family ligands identified to date, with the exception of lymphotoxin-α, are typically type II transmembrane proteins, whose C-terminus is extracellular. In contrast, most receptors in the TNF receptor (TNFR) family identified to date are typically type I transmembrane proteins. In both the TNF ligand and receptor families, however, homology identified between family members has been found mainly in the extracellular domain (“ECD”). Several of the TNF family cytokines, including TNF-α, Apo-1 ligand and CD40 ligand, are cleaved proteolytically at the cell surface; the resulting protein in each case typically forms a homotrimeric molecule that functions as a soluble cytokine. TNF receptor family proteins are also usually cleaved proteolytically to release soluble receptor ECDs that can function as inhibitors of the cognate cytokines.

More recently, other members of the TNFR family have been identified. In von Bulow et al., Science, 278:138-141 (1997), investigators describe a plasma membrane receptor referred to as Transmembrane Activator and CAML-Interactor or “TACI”. The TACI receptor is reported to contain a cysteine-rich motif characteristic of the TNFR family. In an in vitro assay, cross linking of TACI on the surface of transfected Jurkat cells with TACI-specific antibodies led to activation of NF-KB. [see also, WO 98/39361 published Sep. 18, 1998].

Laabi et al., EMBO J., 11:3897-3904 (1992) reported identifying a new gene called “BCM” whose expression was found to coincide with B cell terminal maturation. The open reading frame of the BCM normal cDNA predicted a 184 amino acid long polypeptide with a single transmembrane domain. These investigators later termed this gene “BCMA.” [Laabi et al., Nucleic Acids Res., 22:1147-1154 (1994)]. BCMA mRNA expression was reported to be absent in human malignant B cell lines which represent the pro-B lymphocyte stage, and thus, is believed to be linked to the stage of differentiation of lymphocytes [Gras et al., Int. Immunology, 7:1093-1106 (1995)]. In Madry et al., Int. Immunology, 10:1693-1702 (1998), the cloning of murine BCMA cDNA was described. The murine BCMA cDNA is reported to encode a 185 amino acid long polypeptide having 62% identity to the human BCMA polypeptide. Alignment of the murine and human BCMA protein sequences revealed a conserved motif of six cysteines in the N-terminal region, suggesting that the BCMA protein belongs to the TNFR superfamily [Madry et al., supra].

In Marsters et al., Curr. Biol., 6:750 (1996), investigators describe a full length native sequence human polypeptide, called Apo-3, which exhibits similarity to the TNFR family in its extracellular cysteine-rich repeats and resembles TNFR1 and CD95 in that it contains a cytoplasmic death domain sequence [see also Marsters et al., Curr. Biol., 6:1669 (1996)]. Apo-3 has also been referred to by other investigators as DR3, wsl-1, TRAMP, and LARD [Chinnaiyan et al., Science, 274:990 (1996); Kitson et al., Nature, 384:372 (1996); Bodmer et al., Immunity, 6:79 (1997); Screaton et al., Proc. Natl. Acad. Sci., 94:4615-4619 (1997)].

Pan et al. have disclosed another TNF receptor family member referred to as “DR4” [Pan et al., Science, 276:111-113 (1997); see also WO98/32856 published Jul. 30, 1998]. The DR4 was reported to contain a cytoplasmic death domain capable of engaging the cell suicide apparatus. Pan et al. disclose that DR4 is believed to be a receptor for the ligand known as Apo2L/TRAIL.

In Sheridan et al., Science, 277:818-821 (1997) and Pan et al., Science, 277:815-818 (1997), another molecule believed to be a receptor for Apo2L/TRAIL is described [see also, WO98/51793 published Nov. 19, 1998; WO98/41629 published Sep. 24, 1998]. That molecule is referred to as DR5 (it has also been alternatively referred to as Apo-2; TRAIL-R, TR6, Tango-63, hAPO8, TRICK2 or KILLER [Screaton et al., Curr. Biol., 7:693-696 (1997); Walczak et al., EMBO J., 16:5386-5387 (1997); Wu et al., Nature Genetics, 17:141-143 (1997); WO98/35986 published Aug. 20, 1998; EP870,827 published Oct. 14, 1998; WO98/46643 published Oct. 22, 1998; WO99/02653 published Jan. 21, 1999; WO99/09165 published Feb. 25, 1999; WO99/11791 published Mar. 11, 1999]. Like DR4, DR5 is reported to contain a cytoplasmic death domain and be capable of signaling apoptosis. The crystal structure of the complex formed between Apo-2L/TRAIL and DR5 is described in Hymowitz et al., Molecular Cell, 4:563-571 (1999).

Yet another death domain-containing receptor, DR6, was recently identified [Pan et al., FEBS Letters, 431:351-356 (1998)). Aside from containing four putative extracellular cysteine rich domains and a cytoplasmic death domain, DR6 is believed to contain a putative leucine-zipper sequence that overlaps with a proline-rich motif in the cytoplasmic region. The proline-rich motif resembles sequences that bind to src-homology-3 domains, which are found in many intracellular signal-transducing molecules.

A further group of recently identified receptors are referred to as “decoy receptors,” which are believed to function as inhibitors, rather than transducers of signaling. This group includes DCR1 (also referred to as TRID, LIT or TRAIL-R3) [Pan et al., Science, 276:111-113 (1997); Sheridan et al., Science, 277:818-821 (1997); McFarlane et al., J. Biol. Chem., 272:25417-25420 (1997); Schneider et al., FEBS Letters, 416:329-334 (1997); Degli-Esposti et al., J. Exp. Med., 186:1165-1170 (1997); and Mongkolsapaya et al., J. Immunol., 160:3-6 (1998)] and DCR2 (also called TRUNDD or TRAIL-R4) [Marsters et al., Curr. Biol., 7:1003-1006 (1997); Pan et al., FEBS Letters, 424:41-45 (1998); Degli-Esposti et al., Immunity, 7:813-820 (1997)], both cell surface molecules, as well as OPG [Simonet et al., supra; Emery et al., infra] and DCR3 [Pitti et al., Nature, 396:699-703 (1998)], both of which are secreted, soluble proteins.

Additional newly identified members of the TNFR family include CAR1, HVEM, GITR, ZTNFR-5, NTR-1, and TNFL1 [Brojatsch et al., Cell, 87:845-855 (1996); Montgomery et al., Cell, 87:427-436 (1996); Marsters et al., J. Biol. Chem., 272:14029-14032 (1997); Nocentini et al., Proc. Natl. Acad. Sci. USA 94:6216-6221 (1997); Emery et al., J. Biol. Chem., 273:14363-14367 (1998); WO99/04001 published Jan. 28, 1999; WO99/07738 published Feb. 18, 1999; WO99/33980 published Jul. 8, 1999].

As reviewed recently by Tewari et al., TNFR1, TNFR2 and CD40 modulate the expression of proinflammatory and costimulatory cytokines, cytokine receptors, and cell adhesion molecules through activation of the transcription factor, NF-κB [Tewari et al., Curr. Op. Genet. Develop., 6:39-44 (1996)]. NF-κB is the prototype of a family of dimeric transcription factors whose subunits contain conserved Rel regions [Verma et al., Genes Develop., 9:2723-2735 (1996); Baldwin, Ann. Rev. Immunol., 14:649-681 (1996)]. In its latent form, NF-κB is completed with members of the IκB inhibitor family; upon inactivation of the IκB in response to certain stimuli, released NF-κB translocates to the nucleus where it binds to specific DNA sequences and activates gene transcription. As described above, the TNFR members identified to date either include or lack an intracellular death domain region. Some TNFR molecules lacking a death domain, such as TNFR2, CD40, HVEM, and GITR, are capable of modulating NF-κB activity. [see, e.g., Lotz et al., J. Leukocyte Biol., 60:1-7 (1996)].

For a review of the TNF family of cytokines and their receptors, see Ashkenazi and Dixit, Science, 281:1305-1308 (1998); Golstein, Curr. Biol., 7:750-753 (1997); Gruss and Dower, supra, and Nagata, Cell, 88:355-365 (1997).

### SUMMARY OF THE INVENTION

The recently identified member of the TNF family of molecules called OPGL has been reported to bind at least two receptors, referred to as RANK and OPG. While the expression patterns of this ligand and its receptors, as described in the literature, suggest generically that the interaction(s) of the ligand and receptors may play roles in antigen presenting cell (APC) function(s) and T cell activation, it has not been appreciated in the art what roles OPGL may have in activation of monocytes. Applicants have found that OPGL can activate human monocytes, particularly, in activating such monocytes to secrete certain cytokines such as IL-1 (including IL-1β), IL-6, IL-12, MIP-1α, and TNF-alpha and chemokines such as IL-8. It is also believed that OPGL may function in up-regulation of co-stimulatory molecules such as ICAM-a and VCAM-1, LFA, and B7.1, B7.3, and B7h. OPGL may also serve as an antigen presenting molecule which enhances T cell activation.

The invention thus provides methods of using OPG ligand to activate monocytes, particularly, to activate monocytes to secrete one or more cytokines or chemokines. Optionally, the methods comprise exposing a mammalian cell, such as a peripheral blood monocyte, to OPG ligand in an amount effective to stimulate secretion of one or more cytokines or chemokines by such monocyte. The cell may be in cell culture or in a mammal.

The invention also provides methods of using OPG ligand to treat pathological conditions or diseases in mammals associated with or resulting from lack of, or decreased, cytokine or chemokine secretion by monocytes. In the methods of treatment, OPG ligand may be administered to the mammal suffering from such pathological condition or disease. The OPG ligands contemplated for use in the invention include soluble, extracellular domain sequences of OPG ligand.

The invention further provides agonist and antagonist molecules which can be employed to modulate immune activity, as described herein. Such agonist or antagonist molecules may comprise, for example, antibodies to the OPG or RANK receptors. Agonist RANK antibodies, for instance, may be employed in a manner similar to the OPGL described by the present invention in activating monocytes, particularly, to activate monocytes to secrete one or more cytokines or chemokines. Optionally, the antibody is a monoclonal antibody, chimeric antibody, humanized antibody, antibody fragment or single-chain antibody which specifically binds OPG ligand, OPG receptor or RANK receptor. In one embodiment, the antibody mimics the activity of an OPG ligand polypeptide (an agonist antibody) or conversely the antibody inhibits or neutralizes the activity of an OPG ligand polypeptide (an antagonist antibody). Optionally, the antibody is a monoclonal antibody which preferably has nonhuman complementarity determining region (CDR) residues and human framework region (FR) residues. In a further aspect, the antibody may be an antibody fragment, a single-chain antibody, or an anti-idiotypic antibody.

Compositions employed in the disclosed methods may comprise OPG ligand or other agonist or antagonist and a carrier, such as a pharmaceutically acceptable carrier. Preferably, the composition is sterile. The composition may be employed in the form of a lyophilized formulation or liquid pharmaceutical formulation, which may be preserved to achieve extended storage stability.

In a further embodiment, the invention concerns an article of manufacture, comprising:
(a) a composition of matter comprising OPG ligand polypeptide or other agonist or antagonist;
(b) a container containing said composition; and
(c) a label affixed to said container, or a package insert included in said container referring to the use of said OPG ligand polypeptide or agonist or antagonist in the treatment of a pathological condition, preferably an immune related disease. The composition may comprise a therapeutically effective amount of the OPG ligand polypeptide or the agonist or antagonist.

In particular embodiments of the invention, there are provided methods of stimulating mammalian monocytes, comprising exposing said mammalian monocytes to an effective amount of OPG ligand polypeptide that stimulates said mammalian monocytes to secrete one or more cytokines or chemokines selected from the group consisting of IL-1, IL-6, IL-12, TNF-alpha, MIP-1α, and IL-8, wherein said OPG ligand polypeptide comprises:
a) a polypeptide having at least 80% sequence identity to the full length native sequence OPG ligand polypeptide having the amino acid sequence of FIG. 1B (SEQ ID NO:1);
b) a soluble, extracellular domain sequence of the polypeptide of FIG. 1B (SEQ ID NO:1);
c) a polypeptide consisting of the amino acid sequence of FIG. 1B (SEQ ID NO:1); or
d) a polypeptide comprising a fragment of a), b) or c).
   In the methods, the mammalian monocytes may be exposed to said OPG ligand polypeptide in vitro or in vivo. Optionally, said OPG ligand polypeptide stimulates said mammalian monocytes to secrete IL-1. Optionally, said OPG ligand polypeptide stimulates said mammalian monocytes to secrete IL-6 or IL-12. Optionally, said OPG ligand polypeptide stimulates said mammalian monocytes to secrete TNF-alpha or MIP-1α. Optionally, said OPG ligand polypeptide stimulates said mammalian monocytes to secrete IL-8. Optionally, said OPG ligand polypeptide comprises a soluble, extracellular domain sequence of the polypeptide of FIG. 1B (SEQ ID NO:1). Optionally, said OPG ligand polypeptide has at least 80% sequence identity to the full length native sequence OPG ligand polypeptide having the amino acid sequence of FIG. 1B (SEQ ID NO:1). Optionally, said OPG ligand polypeptide has at least 90% sequence identity.

In further embodiments of the inventions, there are provided methods of stimulating mammalian monocytes, comprising exposing said mammalian monocytes to an effective amount of agonist anti-RANK receptor antibody that stimulates said mammalian monocytes to secrete one or more cytokines or chemokines selected from the group consisting of IL-1, IL-6, IL-12, TNF-alpha, MIP-1α, and IL-8. In the methods, said mammalian monocytes may be exposed to said agonist anti-RANK receptor antibody in vitro or in vivo. Optionally, said agonist anti-RANK receptor antibody stimulates said mammalian monocytes to secrete IL-1. Optionally, said agonist anti-RANK receptor antibody stimulates said mammalian monocytes to secrete IL-6 or IL-12. Optionally, said agonist anti-RANK receptor antibody stimulates said mammalian monocytes to secrete TNF-alpha or MIP-1α. Optionally, said agonist anti-RANK receptor antibody stimulates said mammalian monocytes to secrete IL-8. Optionally, said agonist anti-RANK receptor antibody is a monoclonal antibody. Optionally, said agonist anti-RANK receptor antibody is a chimeric, humanized or human antibody.

In further embodiments of the inventions, there are provided methods of inhibiting mammalian monocytes, comprising exposing said mammalian monocytes to an effective amount of antagonist that inhibits secretion of one or more cytokines or chemokines by said mammalian monocytes, wherein said antagonist comprises an anti-OPG ligand antibody, an anti-OPG receptor antibody, an anti-RANK receptor antibody, an OPG receptor immunoadhesin or a RANK receptor immunoadhesin, and said one or more cytokines or chemokines are selected from the group consisting of IL-1, IL-6, IL-12, TNF-alpha, MIP-1α, and IL-8. In the methods, said mammalian monocytes may be exposed to said antagonist in vitro or in vivo. Optionally, said antagonist inhibits secretion of IL-1 by said mammalian monocytes. Optionally, said antagonist inhibits secretion of IL-6 or IL-12 by said mammalian monocytes. Optionally, said antagonist inhibits secretion of TNF-alpha or MIP-1α by said mammalian monocytes. Optionally, said antagonist inhibits secretion of IL-8 by said mammalian monocytes.

In still further embodiments, there are provided methods of treating a pathological condition associated with or resulting from decreased cytokine or chemokine secretion by mammalian monocytes, comprising administering to a mammal an effective amount of agonist to stimulate the mammal's monocytes to secrete one or more cytokines or chemokines selected from the group consisting of IL-1, IL-6, IL-12, TNF-alpha, MIP-1α, and IL-8, wherein the agonist comprises:
a) a polypeptide having at least 80% sequence identity to the full length native sequence OPG ligand polypeptide having the amino acid sequence of FIG. 1B (SEQ ID NO:1);
b) a soluble, extracellular domain sequence of the polypeptide of FIG. 1B (SEQ ID NO:1);
c) a polypeptide consisting of the amino acid sequence of FIG. 1B (SEQ ID NO:1);
d) a polypeptide comprising a fragment of a), b) or c); or
e) an anti-RANK receptor antibody.
   In the methods, said pathological condition may be an immune related condition. Optionally, said immune related condition is an infectious disease. Optionally, said anti-RANK receptor antibody is a monoclonal antibody. Optionally, said antibody is a chimeric, humanized or human antibody.

In further embodiments, there are provided methods of treating a pathological condition associated with or resulting from increased cytokine or chemokine secretion by mammalian monocytes, comprising administering to a mammal an effective amount of antagonist to inhibit secretion of one or more cytokines or chemokines selected from the group consisting of IL-1, IL-6, IL-12, TNF-alpha, MIP-1α, and IL-8 by said mammal's monocytes, wherein the antagonist comprises an anti-OPG ligand antibody, an anti-OPG receptor antibody, an anti-RANK receptor antibody, an OPG receptor immunoadhesin or a RANK receptor immunoadhesin. In the methods, said pathological condition may be an immune related condition. Optionally, said immune related condition is autoimmune disease, rheumatoid arthritis, insulin dependent diabetes, osteoarthritis, inflammatory bowel disease, psoriasis, transplant rejection or allergy. Optionally, said anti-OPG ligand antibody, anti-OPG receptor antibody, or anti-RANK receptor antibody is a monoclonal antibody. Optionally, said antibody is a chimeric, humanized or human antibody.

In yet additional embodiments of the inventions, there are provided articles of manufacture, comprising:
(a) a composition of matter comprising an effective amount of the OPG ligand polypeptide disclosed herein, agonist disclosed herein, or antagonist disclosed herein;
(b) a container containing said composition; and (c) a label affixed to said container, or a package insert included in said container referring to the use of said OPG ligand polypeptide or agonist or antagonist in the treatment of an immune related disease.

### BRIEF DESCRIPTION OF THE DRAWINGS

FIG. 1A shows the cDNA sequence (SEQ ID NO:2) and FIG. 1B shows the putative amino acid sequence (SEQ ID NO:1) of human OPG ligand.

FIG. 2A shows the cDNA sequence (SEQ ID NO:4) and FIG. 2B shows the putative amino acid sequence (SEQ ID NO:3) of human OPG receptor.

FIG. 3A-1 and 3A-2 show the cDNA sequence (SEQ ID NO:6) and FIG. 3B shows the putative amino acid sequence (SEQ ID NO:5) of human RANK receptor.

FIG. 4 shows the results of an in vitro assay testing the effects of soluble, OPGL on proliferation of monocytes.

FIG. 5 shows the results of an ELISA assay to determine the effects of soluble, OPGL on induction of IL-8 secretion.

FIG. 6 shows the results of an ELISA assay to determine the effects of soluble, OPGL on induction of TNF-alpha secretion.

FIG. 7 shows the results of an ELISA assay to determine the effects of soluble, OPGL on induction of IL-6 secretion.

FIG. 8 shows the results of an ELISA assay to determine the effects of soluble, OPGL on induction of IL-1 secretion.

FIGS. 9A-9E show the results of ELISA assays to determine the effects of OPGL on induction of IL-12, IL-6, TNF-alpha, IL-1beta, and MIP-1alpha secretion.

FIGS. 10A-10H show the results of assays to determine the effects of OPGL on expression of CD80 (10A-10B), Class II (10C-10D), CD86 (10E-10F) and RANK (10G-10H) in monocytes.

FIGS. 11A-11B show the results of assays to examine the effects of OPGL (11A) and OPG receptor (11B) on proliferation of B cells cultured in the presence of IL-4 and/or anti-CD40 antibody.

FIG. 12 shows the results of an assay to determine anti-apoptotic effects of OPGL on monocytes in serum-starved culture.

FIGS. 13A-13B show SDS-PAGE gels which illustrate the effects of OPGL on expression of Bcl-xl (13A) and Bcl-2 (13B) in monocytes treated with OPGL for the indicated number of hours.

FIGS. 14A-14B show SDS-PAGE gels which illustrate the effects of OPGL on expression of p38 MAPK (14A) and p42/44 MAPK (14B) in monocytes treated with OPGL for the indicated number of minutes.

FIG. 15A illustrates the results of FACS analysis of monocytes to detect expression of RANK receptor.

FIG. 15B illustrates the upregulation of RANK mRNA expression in monocytes treated with OPGL, as analyzed by Taqman™ amplification.

FIG. 15C illustrates upregulation of OPGL mRNA expression in normal and ulcerative colitis (“UC”) human tissues, as analyzed by Taqman™ amplification.

### DETAILED DESCRIPTION OF THE INVENTION

I. Definitions

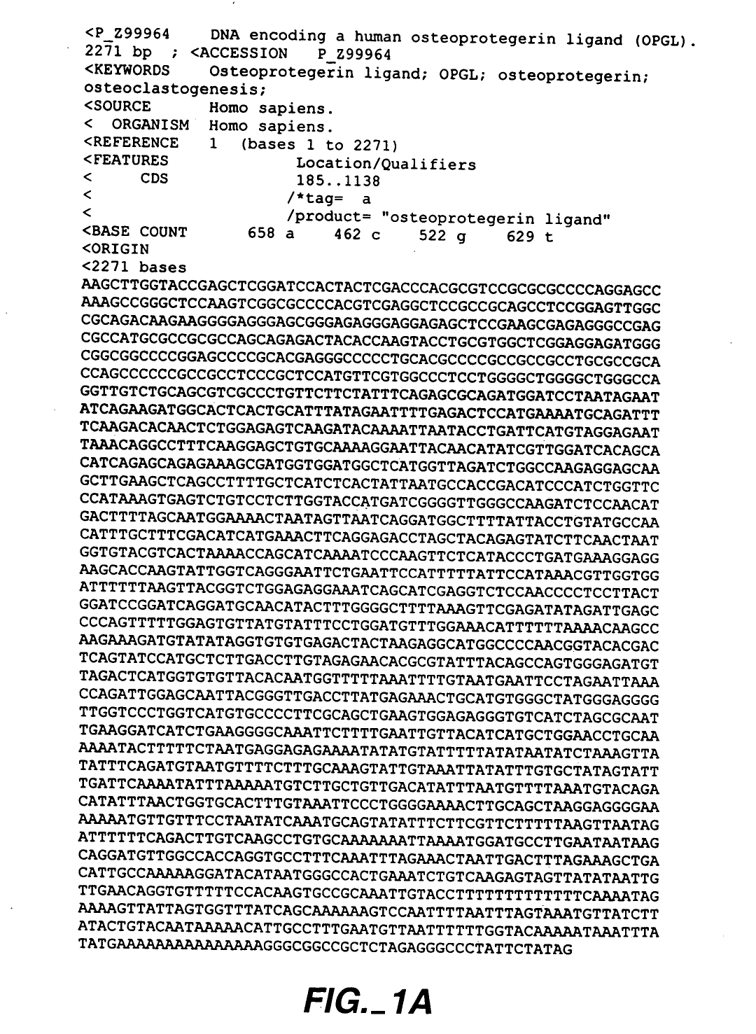

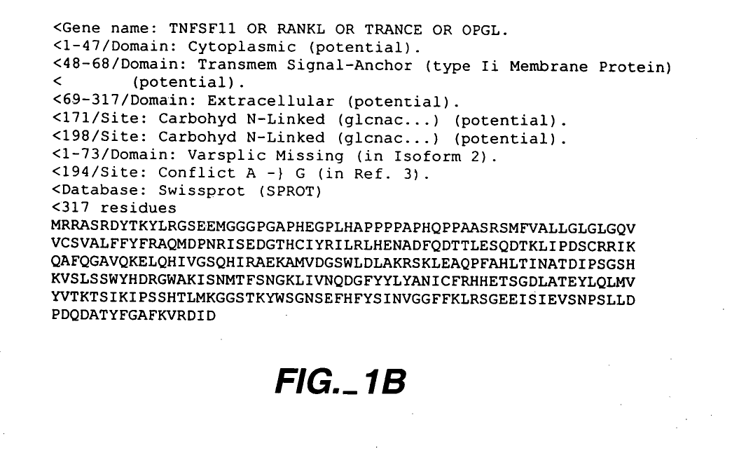

The terms “OPGL” or “OPG Ligand” or “OPG ligand polypeptide” when used herein encompass “native sequence OPGL polypeptides” and “OPGL variants”. “OPGL” is a designation given to those polypeptides which are encoded by the nucleic acid molecules comprising the polynucleotide sequences shown in WO98/28426 published Jul. 2, 1998 (and referred to therein as RANK ligand) and variants thereof, nucleic acid molecules comprising the sequence shown in WO98/28426, and variants thereof as well as fragments of the above which have the biological activity of the native sequence OPGL. Optionally, OPG ligand contemplated for use in the methods includes a polypeptide having the contiguous sequence of amino acid residues 70 to 317 or 1 to 317 of FIG. 1B (SEQ ID NO:1). Variants of OPGL will preferably have at least 80%, more preferably, at least 90%, and even more preferably, at least 95% amino acid sequence identity with the native sequence OPGL polypeptide shown in WO98/28426 and also provided herein in FIG. 1B (SEQ ID NO:1). A “native sequence” OPGL polypeptide comprises a polypeptide having the same amino acid sequence as the corresponding OPGL polypeptide derived from nature. Such native sequence OPGL polypeptides can be isolated from nature or can be produced by recombinant and/or synthetic means. The term “native sequence OPGL polypeptide” specifically encompasses naturally-occurring truncated or secreted forms (e.g., an extracellular domain sequence), naturally-occurring variant forms (e.g., alternatively spliced forms) and naturally-occurring allelic variants of the polypeptide. The term “OPGL” includes those polypeptides described in Anderson et al., Nature, 390:175-179 (1997); Lacey et al., Cell, 93:165-176 (1998); Wong et al., J. Exp. Med., 186:2075-2080 (1997); Yasuda et al., PNAS, 95:3597-3602 (1998); U.S. Pat. No. 6,242,213 issued Jun. 5, 2001; WO99/29865 published Jun. 17, 1999 (referred to as TRANCE). Recombinant human OPG ligand is also commercially available from Alexis Corporation.

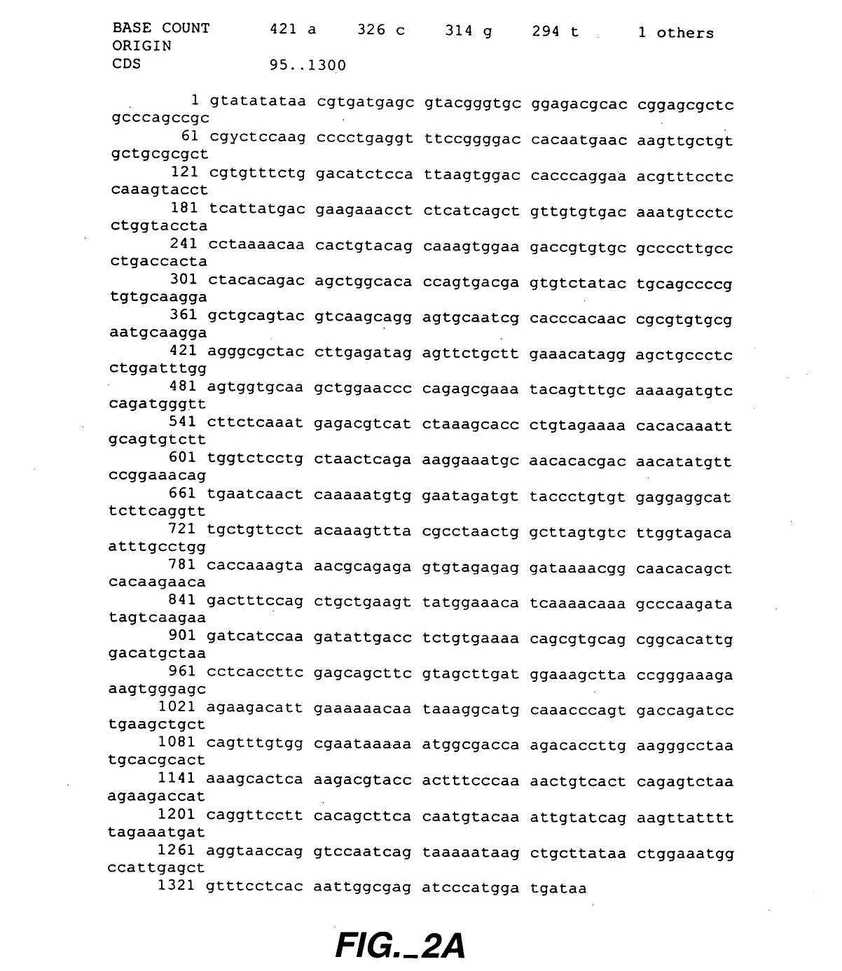

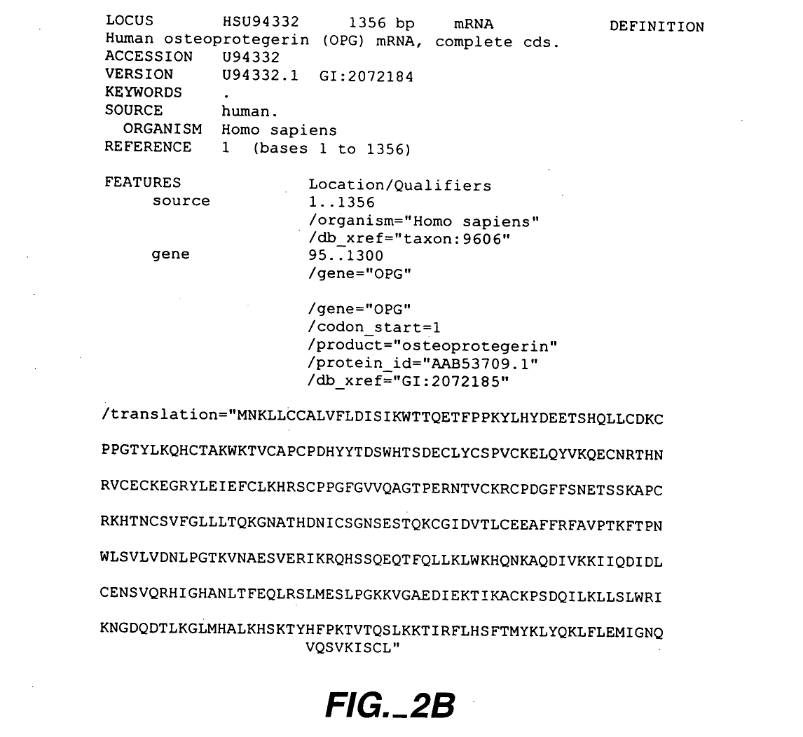

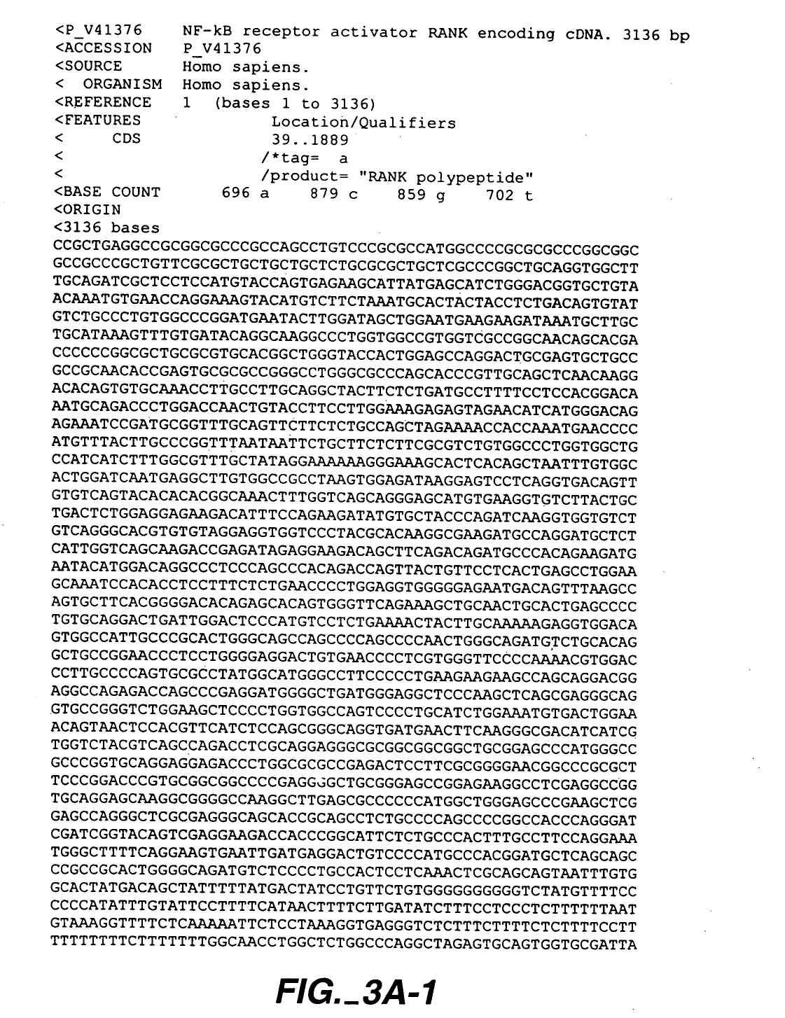

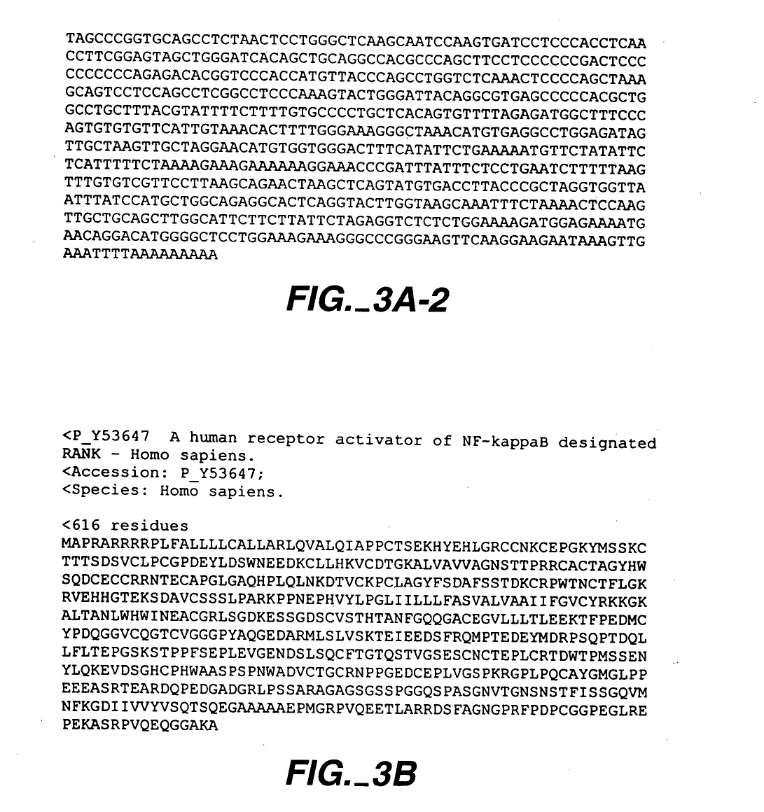

“OPG ligand variant” means an OPG ligand polypeptide having at least about 80% amino acid sequence identity-with the amino acid sequence of a native sequence OPG ligand or OPG ligand ECD. Preferably, the OPG ligand variant binds OPG receptor or RANK receptor, and more preferably, binds to the OPG receptor polypeptide having the amino acid sequence in FIG. 2B (SEQ ID NO:3) or the RANK receptor polypeptide having the amino acid sequence in FIG. 3B (SEQ ID NO:5). Optionally, the OPG ligand variant will have at least one activity identified herein for a native sequence OPG ligand polypeptide or agonist or antagonist molecule. Such OPG ligand variant polypeptides include, for instance, OPG ligand polypeptides wherein one or more amino acid residues are added, or deleted, at the N- and/or C-terminus, as well as within one or more internal domains, of the full-length amino acid sequence. Ordinarily, an OPG ligand variant polypeptide will have at least about 80% amino acid sequence identity, more preferably at least about 81% amino acid sequence identity, more preferably at least about 82% amino acid sequence identity, more preferably at least about 83% amino acid sequence identity, more preferably at least about 84% amino acid sequence identity, more preferably at least about 85% amino acid sequence identity, more preferably at least about 86% amino acid sequence identity, more preferably at least about 87% amino acid sequence identity, more preferably at least about 88% amino acid sequence identity, more preferably at least about 89% amino acid sequence identity, more preferably at least about 90% amino acid sequence identity, more preferably at least about 91% amino acid sequence identity, more preferably at least about 92% amino acid sequence identity, more preferably at least about 93% amino acid sequence identity, more preferably at least about 94% amino acid sequence identity, more preferably at least about 95% amino acid sequence identity, more preferably at least about 96% amino acid sequence identity, more preferably at least about 97% amino acid sequence identity, more preferably at least about 98% amino acid sequence identity and yet more preferably at least about 99% amino acid sequence identity with an OPG ligand polypeptide encoded by a nucleic acid molecule shown in FIG. 1A or a specified fragment thereof. OPG ligand variant polypeptides do not encompass the native OPG ligand polypeptide sequence. Ordinarily, OPG ligand variant polypeptides are at least about 10 amino acids in length, often at least about 20 amino acids in length, more often at least about 30 amino acids in length, more often at least about 40 amino acids in length, more often at least about 50 amino acids in length, more often at least about 60 amino acids in length, more often at least about 70 amino acids in length, more often at least about 80 amino acids in length, more often at least about 90 amino acids in length, more often at least about 100 amino acids in length, more often at least about 150 amino acids in length, more often at least about 200 amino acids in length, more often at least about 250 amino acids in length, more often at least about 300 amino acids in length, or more.

The terms “OPG” or “osteoprotegerin” or “OPG receptor” when used herein encompass “native sequence OPG polypeptides” and “OPG variants” (which are further defined herein). “OPG” is a designation given to those polypeptides which are encoded by the nucleic acid molecules comprising the polynucleotide sequences shown in Simonet et al., Cell, 89:309 (1997) and variants thereof, nucleic acid molecules comprising the sequence shown in Simonet al., supra and variants thereof as well as fragments of the above. The cDNA and putative amino acid sequence is also provided in FIG. 2A-B. Optionally, OPG receptor contemplated for use in the methods includes a polypeptide having the contiguous sequence of amino acid residues 22 to 401 or 1 to 401 of FIG. 2B (SEQ ID NO:3). The OPG polypeptides of the invention may be isolated from a variety of sources, such as from human tissue types or from another source, or prepared by recombinant and/or synthetic methods. A “native sequence” OPG polypeptide comprises a polypeptide having the same amino acid sequence as the corresponding OPG polypeptide derived from nature. Such native sequence OPG polypeptides can be isolated from nature or can be produced by recombinant and/or synthetic means. The term “native sequence OPG polypeptide” specifically encompasses naturally-occurring truncated or secreted forms (e.g., an extracellular domain sequence), naturally-occurring variant forms (e.g., alternatively spliced forms) and naturally-occurring allelic variants of the polypeptide. The OPG polypeptides of the invention include the polypeptides described as “FDCR-1” and “OCIF” in Yasuda et al., Endocrinology, 139:1329 (1998) and Yun et al., J. Immunol., 161:6113-6121 (1998).

“OPG variant” means an OPG polypeptide having at least about 80% amino acid sequence identity with the amino acid sequence of a native sequence OPG or OPG ECD. Preferably, the OPG variant binds OPGL, and more preferably, binds to the full length OPG ligand polypeptide having the amino acid sequence in FIG. 1B (SEQ ID NO:1). Optionally, the OPG variant will have at least one activity identified herein for a native sequence OPG polypeptide or agonist or antagonist molecule. Such OPG variant polypeptides include, for instance, OPG polypeptides wherein one or more amino acid residues are added, or deleted, at the N- and/or C-terminus, as well as within one or more internal domains, of the full-length amino acid sequence. Ordinarily, an OPG variant polypeptide will have at least about 80% amino acid sequence identity, more preferably at least about 81% amino acid sequence identity, more preferably at least about 82% amino acid sequence identity, more preferably at least about 83% amino acid sequence identity, more preferably at least about 84% amino acid sequence identity, more preferably at least about 85% amino acid sequence identity, more preferably at least about 86% amino acid sequence identity, more preferably at least about 87% amino acid sequence identity, more preferably at least about 88% amino acid sequence identity, more preferably at least about 89% amino acid sequence identity, more preferably at least about 90% amino acid sequence identity, more preferably at least about 91% amino acid sequence identity, more preferably at least about 92% amino acid sequence identity, more preferably at least about 93% amino acid sequence identity, more preferably at least about 94% amino acid sequence identity, more preferably at least about 95% amino acid sequence identity, more preferably at least about 96% amino acid sequence identity, more preferably at least about 97% amino acid sequence identity, more preferably at least about 98% amino acid sequence identity and yet more preferably at least about 99% amino acid sequence identity with an OPG polypeptide encoded by a nucleic acid molecule shown in Simonet et al. or a specified fragment thereof. OPG variant polypeptides do not encompass the native OPG polypeptide sequence. Ordinarily, OPG variant polypeptides are at least about 10 amino acids in length, often at least about 20 amino acids in length, more often at least about 30 amino acids in length, more often at least about 40 amino acids in length, more often at least about 50 amino acids in length, more often at least about 60 amino acids in length, more often at least about 70 amino acids in length, more often at least about 80 amino acids in length, more often at least about 90 amino acids in length, more often at least about 100 amino acids in length, more often at least about 150 amino acids in length, more often at least about 200 amino acids in length, more often at least about 250 amino acids in length, more often at least about 300 amino acids in length, or more.

The terms “RANK” or “RANK receptor” when used herein encompass “native sequence RANK polypeptides” and “RANK variants” (which are further defined herein). “RANK” is a designation given to those polypeptides which are encoded by the nucleic acid molecules comprising the polynucleotide sequences shown in WO98/28426 published Jul. 2, 1998 and variants thereof, nucleic acid molecules comprising the sequence shown in WO98/28426 and variants thereof as well as fragments of the above. Optionally, RANK receptor contemplated for use in the methods includes a polypeptide having the contiguous sequence of amino acid residues 29 to 212 or 1 to 212 of FIG. 3B (SEQ ID NO:5). The RANK polypeptides of the invention may be isolated from a variety of sources, such as from human tissue types or from another source, or prepared by recombinant and/or synthetic methods. A “native sequence” RANK polypeptide comprises a polypeptide having the same amino acid sequence as the corresponding RANK polypeptide derived from nature. Such native sequence RANK polypeptides can be isolated from nature or can be produced by recombinant and/or synthetic means. The term “native sequence RANK polypeptide” specifically encompasses naturally-occurring truncated or secreted forms (e.g., an extracellular domain sequence), naturally-occurring variant forms (e.g., alternatively spliced forms) and naturally-occurring allelic variants of the polypeptide. The RANK polypeptides of the invention include the polypeptides described in Anderson et al., Nature, 390:175-179 (1997); U.S. Pat. No. 6,017,729 issued Jan. 25, 2000; and Lacey et al., Cell, 93:165-176 (1998).

“RANK variant” means a RANK polypeptide having at least about 80% amino acid sequence identity with the amino acid sequence of a native sequence RANK or RANK ECD. Preferably, the RANK variant binds OPGL, and more preferably, binds to full length OPG ligand polypeptide having the amino acid sequence in FIG. 1B (SEQ ID NO:1). Optionally, the RANK variant will have at least on activity identified herein for native sequence RANK polypeptide or agonist or antagonist molecule. Such RANK variant polypeptides include, for instance, RANK polypeptides wherein one or more amino acid residues are added, or deleted, at the N- and/or C-terminus, as well as within one or more internal domains, of the full-length amino acid sequence. Ordinarily, a RANK variant polypeptide will have at least about 80% amino acid sequence identity, more preferably at least about 81% amino acid sequence identity, more preferably at least about 82% amino acid sequence identity, more preferably at least about 83% amino acid sequence identity, more preferably at least about 84% amino acid sequence identity, more preferably at least about 85% amino acid sequence identity, more preferably at least about 86% amino acid sequence identity, more preferably at least about 87% amino acid sequence identity, more preferably at least about 88% amino acid sequence identity, more preferably at least about 89% amino acid sequence identity, more preferably at least about 90% amino acid sequence identity, more preferably at least about 91% amino acid sequence identity, more preferably at least about 92% amino acid sequence identity, more preferably at least about 93% amino acid sequence identity, more preferably at least about 94% amino acid sequence identity, more preferably at least about 95% amino acid sequence identity, more preferably at least about 96% amino acid sequence identity, more preferably at least about 97% amino acid sequence identity, more preferably at least about 98% amino acid sequence identity and yet more preferably at least about 99% amino acid sequence identity with a RANK polypeptide encoded by a nucleic acid molecule shown in WO98/28426 or a specified fragment thereof. RANK variant polypeptides do not encompass the native RANK polypeptide sequence. Ordinarily, RANK variant polypeptides are at least about 10 amino acids in length, often at least about 20 amino acids in length, more often at least about 30 amino acids in length, more often at least about 40 amino acids in length, more often at least about 50 amino acids in length, more often at least about 60 amino acids in length, more often at least about 70 amino acids in length, more often at least about 80 amino acids in length, more often at least about 90 amino acids in length, more often at least about 100 amino acids in length, more often at least about 150 amino acids in length, more often at least about 200 amino acids in length, more often at least about 250 amino acids in length, more often at least about 300 amino acids in length, or more.

An “extracellular domain” or “ECD” refers to a form of the polypeptide which is essentially free of the transmembrane and cytoplasmic domains. Ordinarily, an ECD form of a polypeptide will have less than about 1% of such transmembrane and/or cytoplasmic domains and preferably, will have less than about 0.5% of such domains. It will be understood that any transmembrane domain(s) identified for the polypeptides of the present invention are identified pursuant to criteria routinely employed in the art for identifying that type of hydrophobic domain. The exact boundaries of a transmembrane domain may vary but most likely by no more than about 5 amino acids at either end of the domain as initially identified. In a preferred embodiment, the ECD will consist of a soluble, extracellular domain sequence of the polypeptide which is free of the transmembrane and cytoplasmic or intracellular domains (and is not membrane bound).

“Percent (%) amino acid sequence identity”0 with respect to the ligand or receptor polypeptide sequences identified herein is defined as the percentage of amino acid residues in a candidate sequence that are identical with the amino acid residues in such a ligand or receptor sequence identified herein, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, ALIGN-2 or Megalign (DNASTAR) software. Those skilled in the art can determine appropriate parameters for measuring alignment, including any algorithms needed to achieve maximal alignment over the full-length of the sequences being compared. For purposes herein, however, % amino acid sequence identity values are obtained as described below by using the sequence comparison computer program ALIGN-2. The ALIGN-2 sequence comparison computer program was authored by Genentech, Inc. and the source code has been filed with user documentation in the U.S. Copyright Office, Washington D.C., 20559, where it is registered under U.S. Copyright Registration No. TXU510087. The ALIGN-2 program is publicly available through Genentech, Inc., South San Francisco, Calif. The ALIGN-2 program should be compiled for use on a UNIX operating system, preferably digital UNIX V4.0D. All sequence comparison parameters are set by the ALIGN-2 program and do not vary.

For purposes herein, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:
100 times the fraction X/Y
where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described above using the ALIGN-2 sequence comparison computer program. However, % amino acid sequence identity may also be determined using the sequence comparison program NCBI-BLAST2 (Altschul et al., Nucleic Acids Res. 25:3389-3402 (1997)). The NCBI-BLAST2 sequence comparison program may be downloaded from the NCBI internet web site. NCBI-BLAST2 uses several search parameters, wherein all of those search parameters are set to default values including, for example, unmask=yes, strand=all, expected occurrences=10, minimum low complexity length=15/5, multi-pass e-value=0.01, constant for multi-pass=25, dropoff for final gapped alignment=25 and scoring matrix=BLOSUM62.

In situations where NCBI-BLAST2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:
100 times the fraction X/Y
where X is the number of amino acid residues scored as identical matches by the sequence alignment program NCBI-BLAST2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to re-anneal when complementary strands are present in an environment below their melting temperature. The higher the degree of desired identity between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent conditions” or “high stringency conditions”, as defined herein, may be identified by those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C.

“Moderately stringent conditions” may be identified as described by Sambrook et al., Molecular Cloning: A Laboratory Manual, New York: Cold Spring Harbor Press, 1989, and include the use of washing solution and hybridization conditions (e.g., temperature, ionic strength and %SDS) less stringent that those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5×Denhardt's solution, 10% dextran sulfate, and 20 mg/ml denatured sheared salmon sperm DNA, followed by washing the filters in 1×SSC at about 37-50° C. The skilled artisan will recognize how to adjust the temperature, ionic strength, etc. as necessary to accommodate factors such as probe length and the like.

The term “epitope tagged” when used herein refers to a chimeric polypeptide comprising a polypeptide fused to a “tag polypeptide”. The tag polypeptide has enough residues to provide an epitope against which an antibody can be made. The tag polypeptide preferably also is fairly unique so that the antibody does not substantially cross-react with other epitopes. Suitable tag polypeptides generally have at least six amino acid residues and usually between about 8 and 50 amino acid residues (preferably, between about 10 and 20 amino acid residues).

As used herein, the term “immunoadhesin” designates antibody-like molecules which combine the binding specificity of a heterologous protein (an “adhesin”) with the effector functions of immunoglobulin constant domains. Structurally, the immunoadhesins comprise a fusion of an amino acid sequence with the desired binding specificity which is other than the antigen recognition and binding site of an antibody (i.e., is “heterologous”), and an immunoglobulin constant domain sequence. The adhesin part of an immunoadhesin molecule typically is a contiguous amino acid sequence comprising at least the binding site of a receptor or a ligand. The immunoglobulin constant domain sequence in the immunoadhesin may be obtained from any immunoglobulin, such as IgG-1, IgG-2, IgG-3, or IgG-4 subtypes, IgA (including IgA-1 and IgA-2), IgE, IgD or IgM.

The term “antagonist” is used in the broadest sense, and includes any molecule that partially or fully blocks, inhibits, or neutralizes one or more biological activities of OPGL, in vitro, in situ, or in vivo. Examples of such biological activities of OPGL polypeptides include binding of OPGL to OPG or RANK, proliferation of B cells, and activation of monocytes, particularly stimulating cytokine or chemokine secretion by monocytes. An antagonist may function in a direct or indirect manner. For instance, the antagonist may function to partially or fully block, inhibit or neutralize one or more biological activities of OPGL, in vitro, in situ, or in vivo as a result of its direct binding to OPGL, OPG or RANK. The antagonist may also function indirectly to partially or fully block, inhibit or neutralize one or more biological activities of OPGL, in vitro, in situ, or in vivo as a result of, e.g., blocking or inhibiting another effector molecule.

The term “agonist” is used in the broadest sense, and includes any molecule that mimics or functions similarly to OPGL, and preferably, partially or fully enhances, stimulates or activates one or more biological activities of OPG or RANK, in vitro, in situ, or in vivo. Examples of such biological activities of OPGL include proliferation of B cells and activation of monocytes, particularly stimulating cytokine or chemokine secretion by such monocytes. An agonist may function in a direct or indirect manner. For instance, the agonist may function to partially or fully enhance, stimulate or activate one or more biological activities of OPG or RANK, in vitro, in situ, or in vivo as a result of its direct binding to OPG or RANK, which causes receptor activation or signal transduction. The agonist may also function indirectly to partially or fully enhance, stimulate or activate one or more biological activities of OPG or RANK, in vitro, in situ, or in vivo as a result of, e.g., stimulating another effector molecule which then causes OPG or RANK receptor activation or signal transduction.

The term “OPGL antagonist” refers to any molecule that partially or fully blocks, inhibits, or neutralizes a biological activity of OPGL and includes, but are not limited to, soluble forms of OPG receptor or RANK receptor such as an extracellular domain sequence of OPG or RANK, OPG receptor immunoadhesins, RANK receptor immunoadhesins, OPG receptor fusion proteins, RANK receptor fusion proteins, covalently modified forms of OPG receptor, covalently modified forms of RANK receptor, OPG variants, RANK variants, OPG receptor antibodies, RANK receptor antibodies, and OPGL antibodies. To determine whether an OPGL antagonist molecule partially or fully blocks, inhibits or neutralizes a biological activity of OPGL, assays may be conducted to assess the effect(s) of the antagonist molecule on, for example, binding of OPGL to OPG or to RANK, or monocyte activation by the OPGL. Such assays may be conducted in known in vitro or in vivo assay formats, for instance, in cells expressing OPG and/or RANK. Preferably, the OPGL antagonist employed in the methods described herein will be capable of blocking or neutralizing at least one type of OPGL activity, which may optionally be determined in assays such as described herein (and in the Examples). Optionally, an antagonist will be capable of reducing or inhibiting binding of OPGL to OPG or to RANK by at least 50%, preferably, by at least 90%, more preferably by at least 99%, and most preferably, by 100%, as compared to a negative control molecule, in a binding assay. In one embodiment, the antagonist will comprise antibodies which will competitively inhibit the binding of OPGL to OPG or RANK. Methods for determining antibody specificity and affinity by competitive inhibition are known in the art [see, e.g., Harlow et al., Antibodies:A Laboratory Manual, Cold Spring Harbor Laboratory Press, Cold Spring Harbor, N.Y. (1998); Colligan et al., Current Protocols in Immunology, Green Publishing Assoc., NY (1992; 1993); Muller, Meth. Enzym., 92:589-601 (1983)].

The term “agonist” refers to any molecule that partially or fully enhances, stimulates or activates a biological activity of OPG or RANK, respectively, or both OPG and RANK, and include, but are not limited to, anti-OPG receptor antibodies and anti-RANK receptor antibodies. To determine whether a RANK agonist molecule partially or fully enhances, stimulates, or activates a biological activity of RANK, assays may be conducted to assess the effect(s) of the agonist molecule on, for example, monocytes or OPG or RANK-transfected cells. Such assays may be conducted in known in vitro or in vivo assay formats. Preferably, the RANK agonist employed in the methods described herein will be capable of enhancing or activating at least one type of RANK activity, which may optionally be determined in assays such as described herein. Preferably, the OPG agonist or RANK agonist will be capable of stimulating or activating OPG or RANK, respectively, to the extent of that accomplished by the native ligand (OPGL) for the OPG or RANK receptors.

The term “antibody” is used in the broadest sense and specifically covers, for example, single monoclonal antibodies which specifically bind OPGL, RANK or OPG, antibody compositions with polyepitopic specificity, single chain antibodies, and fragments of antibodies.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature, 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature, 352:624-628 (1991) and Marks et al., J. Mol. Biol., 222:581-597 (1991), for example.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855 (1984)). Methods of making chimeric antibodies are known in the art.

“Humanized” forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. For the most part, humanized antibodies are human immunoglobulins (recipient antibody) in which residues from a complementarity-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, humanized antibodies may comprise residues which are found neither in the recipient antibody nor in the imported CDR or framework sequences. These modifications are made to further refine and maximize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody optimally also will comprise at least a portion of an immunoglobulin constant region (Fc), typically that of a human immunoglobulin. For further details, see Jones et al., Nature, 321:522-525 (1986); Reichmann et al., Nature, 332:323-329 (1988); and Presta, Curr. Op. Struct. Biol., 2:593-596 (1992). The humanized antibody includes a PRIMATIZED™ antibody wherein the antigen-binding region of the antibody is derived from an antibody produced by immunizing macaque monkeys with the antigen of interest. Methods of making humanized antibodies are known in the art.

Human antibodies can also be produced using various techniques known in the art, including phage-display libraries. Hoogenboom and Winter, J. Mol. Biol., 227:381 (1991); Marks et al., J. Mol. Biol., 222:581 (1991). The techniques of Cole et al. and Boerner et al. are also available for the preparation of human monoclonal antibodies. Cole et al., Monoclonal Antibodies and Cancer Therapy, Alan R. Liss, p. 77 (1985); Boerner et al., J. Immunol., 147(1):86-95 (1991).

“Antibody fragments” comprise a portion of an intact antibody, preferably the antigen binding or variable region of the intact antibody. Examples of antibody fragments include Fab, Fab′, F(ab′)₂, and Fv fragments; diabodies; linear antibodies (Zapata et al., Protein Eng. 8(10): 1057-1062[1995]); single-chain antibody molecules; and multispecific antibodies formed from antibody fragments.

Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)₂ fragment that has two antigen-combining sites and is still capable of cross-linking antigen.

“Fv” is the minimum antibody fragment which contains a complete antigen-recognition and -binding site. This region consists of a dimer of one heavy- and one light-chain variable domain in tight, non-covalent association. It is in this configuration that the three CDRs of each variable domain interact to define an antigen-binding site on the surface of the V_{H}-V_{L} dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The Fab fragment also contains the constant domain of the light chain and the first constant domain (CH1) of the heavy chain. Fab fragments differ from Fab′ fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The “light chains” of antibodies (immunoglobulins) from any vertebrate species can be assigned to one of two clearly distinct types, called kappa and lambda, based on the amino acid sequences of their constant domains.

Depending on the amino acid sequence of the constant domain of their heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA, and IgA2.

“Single-chain Fv” or “sFv” antibody fragments comprise the V_{H} and V_{L} domains of antibody, wherein these domains are present in a single polypeptide chain. Preferably, the Fv polypeptide further comprises a polypeptide linker between the V_{H} and V_{L} domains which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (V_{H}) connected to a light-chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

An antibody that “specifically binds to” or is “specific for” a particular polypeptide or an epitope on a particular polypeptide is one that binds to that particular polypeptide or epitope on a particular polypeptide without substantially binding to any other polypeptide or polypeptide epitope.

“Isolated,” when used to describe the various proteins disclosed herein, means protein that has been identified and separated and/or recovered from a component of its natural environment. Contaminant components of its natural environment are materials that would typically interfere with diagnostic or therapeutic uses for the protein, and may include enzymes, hormones, and other proteinaceous or non-proteinaceous solutes. In preferred embodiments, the protein will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Isolated protein includes protein in situ within recombinant cells, since at least one component of the protein natural environment will not be present. Ordinarily, however, isolated protein will be prepared by at least one purification step.

The term “cytokine” is a generic term for proteins released by one cell population which act on another cell as intercellular mediators. Examples of such cytokines are lymphokines, monokines, and traditional polypeptide hormones. Included among the cytokines are growth hormone such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-α and -β; mullerian-inhibiting substance; mouse gonadotropin-associated peptide; inhibin; activin; vascular endothelial growth factor; integrin; thrombopoietin (TPO); nerve growth factors; platelet-growth factor; transforming growth factors (TGFs) such as TGF-α and TGF-β; insulin-like growth factor-I and -II; erythropoietin (EPO); osteoinductive factors; interferons such as interferon-α, -β and -gamma; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-2, IL-3, IL-4, IL-5, IL-6, IL-7, IL-8, IL-9, IL-11, IL-12; and other polypeptide factors including LIF, MIP-1α, and kit ligand (KL). As used herein, the term cytokine includes proteins from natural sources or from recombinant cell culture and biologically active equivalents of the native sequence cytokines.

A “liposome” is a small vesicle composed of various types of lipids, phospholipids and/or surfactant which is useful for delivery of a drug (such as a polypeptide or antibody thereto) to a mammal. The components of the liposome are commonly arranged in a bilayer formation, similar to the lipid arrangement of biological membranes.

A “small molecule” is defined herein to have a molecular weight below about 500 Daltons.

The term “immune related disease” means a disease in which a component of the immune system of a mammal causes, mediates or otherwise contributes to a morbidity in the mammal. Also included are diseases in which stimulation or intervention of the immune response has an ameliorative effect on progression of the disease. Included within this term are immune-mediated inflammatory diseases, non-immune-mediated inflammatory diseases, infectious diseases, immunodeficiency diseases, and neoplasia.

The term “T cell mediated disease” means a disease in which T cells directly or indirectly mediate or otherwise contribute to a morbidity in a mammal. The T cell mediated disease may be associated with cell mediated effects, lymphokine mediated effects, etc., and even effects associated with B cells if the B cells are stimulated, for example, by the lymphokines secreted by T cells.

Examples of immune-related and inflammatory diseases, some of which are immune or T cell mediated, which can be treated according to the invention include systemic lupus erythematosis, rheumatoid arthritis, juvenile chronic arthritis, spondyloarthropathies, systemic sclerosis (scleroderma), idiopathic inflammatory myopathies (dermatomyositis, polymyositis), Sjogren's syndrome, systemic vasculitis, sarcoidosis, autoimmune hemolytic anemia (immune pancytopenia, paroxysmal nocturnal hemoglobinuria), autoimmune thrombocytopenia (idiopathic thrombocytopenic purpura, immune-mediated thrombocytopenia), thyroiditis (Grave's disease, Hashimoto's thyroiditis, juvenile lymphocytic thyroiditis, atrophic thyroiditis), diabetes mellitus, immune-mediated renal disease (glomerulonephritis, tubulointerstitial nephritis), demyelinating diseases of the central and peripheral nervous systems such as multiple sclerosis, idiopathic demyelinating polyneuropathy or Guillain-Barre syndrome, and chronic inflammatory demyelinating polyneuropathy, hepatobiliary diseases such as infectious hepatitis (hepatitis A, B, C, D, E and other non-hepatotropic viruses), autoimmune chronic active hepatitis, primary biliary cirrhosis, granulomatous hepatitis, and sclerosing cholangitis, inflammatory bowel disease (ulcerative colitis; Crohn's disease), gluten-sensitive enteropathy, and Whipple's disease, autoimmune or immune-mediated skin diseases including bullous skin diseases, erythema multiforme and contact dermatitis, psoriasis, allergic diseases such as asthma, allergic rhinitis, atopic dermatitis, food hypersensitivity and urticaria, immunologic diseases of the lung such as eosinophilic pneumonias, idiopathic pulmonary fibrosis and hypersensitivity pneumonitis, transplantation associated diseases including graft rejection and graft-versus-host-disease. Infectious diseases including viral diseases such as AIDS (HIV infection), hepatitis A, B, C, D, and E, herpes, etc., bacterial infections, fungal infections, protozoal infections and parasitic infections.

The term “effective amount” is a concentration or amount of an OPGL polypeptide and/or agonist/antagonist which results in achieving a particular stated purpose. An “effective amount” of an OPGL polypeptide or agonist or antagonist thereof may be determined empirically. Furthermore, a “therapeutically effective amount” is a concentration or amount of an OPGL polypeptide and/or agonist/antagonist which is effective for achieving a stated therapeutic effect. This amount may also be determined empirically.

The term “cytotoxic agent” as used herein refers to a substance that inhibits or prevents the function of cells and/or causes destruction of cells. The term is intended to include radioactive isotopes (e.g., I¹³¹, I¹²⁵, Y⁹⁰ and Re¹⁸⁶), chemotherapeutic agents, and toxins such as enzymatically active toxins of bacterial, fungal, plant or animal origin, or fragments thereof.

A “chemotherapeutic agent” is a chemical compound useful in the treatment of cancer. Examples of chemotherapeutic agents include alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin gammalI and calicheamicin phiIl, see, e.g., Agnew, Chem Intl. Ed. Engl., 33:183-186 (1994); dynemicin, including dynemicin A; bisphosphonates, such as clodronate; an esperamicin; as well as neocarzinostatin chromophore and related chromoprotein enediyne antiobiotic chromomophores), aclacinomysins, actinomycin, authramycin, azaserine, bleomycins, cactinomycin, carabicin, carminomycin, carzinophilin, chromomycins, dactinomycin, daunorubicin, detorubicin, 6-diazo-5-oxo-L-norleucine, doxorubicin (Adriamycin™) (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; losoxantrone; podophyllinic acid; 2-ethylhydrazide; procarbazine; PSK®; razoxane; rhizoxin; sizofiran; spirogermanium; tenuazonic acid; triaziquone; 2, 2′, 2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.J.) and doxetaxel (TAXOTERE®, Rhône-Poulenc Rorer, Antony, France); chlorambucil; gemcitabine (Gemzar™); 6-thioguanine; mercaptopurine; methotrexate; platinum analogs such as cisplatin and carboplatin; vinblastine; platinum; etoposide (VP-16); ifosfamide; mitoxantrone; vincristine; vinorelbine (Navelbine™); novantrone; teniposide; edatrexate; daunomycin; aminopterin; xeloda; ibandronate; CPT-11; topoisomerase inhibitor RFS 2000; difluoromethylornithine (DMFO); retinoids such as retinoic acid; capecitabine; and pharmaceutically acceptable salts, acids or derivatives of any of the above. Also included in this definition are anti-hormonal agents that act to regulate or inhibit hormone action on tumors such as anti-estrogens and selective estrogen receptor modulators (SERMs), including, for example, tamoxifen (including Nolvadex™), raloxifene, droloxifene, 4-hydroxytamoxifen, trioxifene, keoxifene, LY117018, onapristone, and toremifene (Fareston™); aromatase inhibitors that inhibit the enzyme aromatase, which regulates estrogen production in the adrenal glands, such as, for example, 4(5)-imidazoles, aminoglutethimide, megestrol acetate (Megace™), exemestane, formestane, fadrozole, vorozole (Rivisor™), letrozole (Femara™), and anastrozole (Arimidex™); and anti-androgens such as flutamide, nilutamide, bicalutamide, leuprolide, and goserelin; and pharmaceutically acceptable salts, acids or derivatives of any of the above.

A “growth inhibitory agent” when used herein refers to a compound or composition which inhibits growth of a cell, especially cancer cell overexpressing any of the genes identified herein, either in vitro or in vivo. Thus, the growth inhibitory agent is one which significantly reduces the percentage of cells overexpressing such genes in S phase. Examples of growth inhibitory agents include agents that block cell cycle progression (at a place other than S phase), such as agents that induce G1 arrest and M-phase arrest. Classical M-phase blockers include the vincas (vincristine and vinblastine), taxol, and topo II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in The Molecular Basis of Cancer, Mendelsohn and Israel, eds., Chapter 1, entitled “Cell cycle regulation, oncogens, and antineoplastic drugs” by Murakami et al. (WB Saunders: Philadelphia, 1995), especially p. 13.

The term “monocyte” as used herein refers to a mammalian cell which is characterized as being a mononuclear cell that has the potential to differentiate into a resident macrophage. The term monocyte is used herein in a general sense and includes but is not limited to monoblasts and promonocytes. Monocytes are typically Class II MHC cells and typically express markers known in the art as CD14, CD62, CD32, and CD16. In vivo, monocytes typically circulate in the blood and bone marrow. Monocytes may function, for example, in phagocytosis, antigen presentation, and secretion of molecules like metalloproteases, nitric oxide, and certain chemokines.

“Treatment” or “therapy” refer to both therapeutic treatment and prophylactic or preventative measures.

“Chronic” administration refers to administration of the agent(s) in a continuous mode as opposed to an acute mode, so as to maintain the initial therapeutic effect (activity) for an extended period of time. “Intermittent” administration is treatment that is not consecutively done without interruption, but rather is cyclic in nature.

Administration “in combination with” one or more further therapeutic agents includes simultaneous (concurrent) and consecutive administration in any order.

“Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

“Mammal” for purposes of treatment or therapy refers to any animal classified as a mammal, including humans, domestic and farm animals, and zoo, sports, or pet animals, such as dogs, horses, cats, cows, etc. Preferably, the mammal is human.

II. Methods and Materials

Applicants have surprisingly found that OPG ligand can activate monocytes to secrete various cytokines and chemokines. Exposing mammalian cells, such as monocytes, to an effective amount of OPG ligand, or an agonist molecule which mimics the activity of OPG ligand, can be useful for a variety of applications. For instance, increasing secretion of cytokines like IL-1, IL-6, IL-8, IL-12, MIP-1α, or TNF-alpha will be useful for proinflammatory purposes, particularly in vivo to treat infection (like parasitic infection or microbial infection). Increasing secretion of cytokines like IL-1, IL-6, IL-8, IL-12, MIP-1α or TNF-alpha may also be useful in enhancing T cell activation, activation of natural killer (NK) cells or antibody dependent cytotoxicity (ADCC). Increased secretion of such cytokines further finds utility in cancer treatments to assist in inhibiting or decreasing tumor growth.

Inhibition or neutralization of the activity of OPG ligand will also be useful in the methods described herein for employing antagonist molecules. Antagonist molecules which inhibit or decrease secretion of such cytokines or chemokines may be useful in the treatment of conditions such as autoimmune disease, rheumatoid arthritis, insulin dependent diabetes, osteoarthritis, inflammatory bowel disease (such as ulcerative colitis or Crohn's disease), psoriasis, transplant rejection or allergic responses.

A. Materials

The OPGL polypeptide which can be employed in the methods include, but are not limited to, soluble forms of OPGL, fusion proteins comprising OPGL, covalently modified forms of OPGL, and OPGL variants. Antagonist or agonist molecules may also be employed. Various techniques that can be employed for making such compositions are described below.

Generally, the compositions of the invention may be prepared using recombinant techniques known in the art. The description below relates to methods of producing such polypeptides by culturing host cells transformed or transfected with a vector containing the encoding nucleic acid and recovering the polypeptide from the cell culture. (See, e.g., Sambrook et al., Molecular Cloning: A Laboratory Manual (New York: Cold Spring Harbor Laboratory Press, 1989); Dieffenbach et al., PCR Primer:A Laboratory Manual (Cold Spring Harbor Laboratory Press, 1995)).

The nucleic acid (e.g., cDNA or genomic DNA) encoding the desired polypeptide may be inserted into a replicable vector for further cloning (amplification of the DNA) or for expression. Various vectors are publicly available. The vector components generally include, but are not limited to, one or more of the following: a signal sequence, an origin of replication, one or more marker genes, an enhancer element, a promoter, and a transcription termination sequence, each of which is described below. Optional signal sequences, origins of replication, marker genes, enhancer elements and transcription terminator sequences that may be employed are known in the art and described in further detail in WO97/25428.

Expression and cloning vectors usually contain a promoter that is recognized by the host organism and is operably linked to the encoding nucleic acid sequence. Promoters are untranslated sequences located upstream (5′) to the start codon of a structural gene (generally within about 100 to 1000 bp) that control the transcription and translation of a particular nucleic acid sequence, to which they are operably linked. Such promoters typically fall into two classes, inducible and constitutive. Inducible promoters are promoters that initiate increased levels of transcription from DNA under their control in response to some change in culture conditions, e.g., the presence or absence of a nutrient or a change in temperature. At this time a large number of promoters recognized by a variety of potential host cells are well known. These promoters are operably linked to the encoding DNA by removing the promoter from the source DNA by restriction enzyme digestion and inserting the isolated promoter sequence into the vector.

Promoters suitable for use with prokaryotic and eukaryotic hosts are known in the art, and are described in further detail in WO97/25428.

Construction of suitable vectors containing one or more of the above-listed components employs standard ligation techniques. Isolated plasmids or DNA fragments are cleaved, tailored, and re-ligated in the form desired to generate the plasmids required. For analysis to confirm correct sequences in plasmids constructed, the ligation mixtures can be used to transform E. coli K12 strain 294 (ATCC 31,446) and successful transformants selected by ampicillin or tetracycline resistance where appropriate. Plasmids from the transformants are prepared, analyzed by restriction endonuclease digestion, and/or sequenced using standard techniques known in the art. [See, e.g., Messing et al., Nucleic Acids Res., 9:309 (1981); Maxam et al., Methods in Enzymology, 65:499 (1980)).

Expression vectors that provide for the transient expression in mammalian cells of the encoding DNA may be employed. In general, transient expression involves the use of an expression vector that is able to replicate efficiently in a host cell, such that the host cell accumulates many copies of the expression vector and, in turn, synthesizes high levels of a desired polypeptide encoded by the expression vector [Sambrook et al., supra]. Transient expression systems, comprising a suitable expression vector and a host cell, allow for the convenient positive identification of polypeptides encoded by cloned DNAs, as well as for the rapid screening of such polypeptides for desired biological or physiological properties.

Other methods, vectors, and host cells suitable for adaptation to the synthesis of the desired polypeptide in recombinant vertebrate cell culture are described in Gething et al., Nature, 293:620-625 (1981); Mantei et al., Nature, 281:40-46 (1979); EP 117,060; and EP 117,058.

Suitable host cells for cloning or expressing the DNA in the vectors herein include prokaryote, yeast, or higher eukaryote cells. Suitable prokaryotes for this purpose include but are not limited to eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710 published 12 Apr. 1989), Pseudomonas such as P. aeruginosa, and Streptomyces. Preferably, the host cell should secrete minimal amounts of proteolytic enzymes.

In addition to prokaryotes, eukaryotic microbes such as filamentous fungi or yeast are suitable cloning or expression hosts for vectors. Suitable host cells for the expression of glycosylated polypeptide are derived from multicellular organisms. Examples of all such host cells are described further in WO97/25428.

Host cells are transfected and preferably transformed with the above-described expression or cloning vectors and cultured in nutrient media modified as appropriate for inducing promoters, selecting transformants, or amplifying the genes encoding the desired sequences.

Transfection refers to the taking up of an expression vector by a host cell whether or not any coding sequences are in fact expressed. Numerous methods of transfection are known to the ordinarily skilled artisan, for example, CaPO₄ and electroporation. Successful transfection is generally recognized when any indication of the operation of this vector occurs within the host cell.

Transformation means introducing DNA into an organism so that the DNA is replicable, either as an extrachromosomal element or by chromosomal integrant. Depending on the host cell used, transformation is done using standard techniques appropriate to such cells. The calcium treatment employing calcium chloride, as described in Sambrook et al., supra, or electroporation is generally used for prokaryotes or other cells that contain substantial cell-wall barriers. Infection with Agrobacterium tumefaciens is used for transformation of certain plant cells, as described by Shaw et al., Gene, 23:315 (1983) and WO 89/05859 published 29 Jun. 1989. In addition, plants may be transfected using ultrasound treatment as described in WO 91/00358 published 10 Jan. 1991.

For mammalian cells without such cell walls, the calcium phosphate precipitation method of Graham and van der Eb, Virology, 52:456-457 (1978) may be employed. General aspects of mammalian cell host system transformations have been described in U.S. Pat. No. 4,399,216. Transformations into yeast are typically carried out according to the method of Van Solingen et al., J. Bact., 130:946 (1977) and Hsiao et al., Proc. Natl. Acad. Sci. (USA), 76:3829 (1979). However, other methods for introducing DNA into cells, such as by nuclear microinjection, electroporation, bacterial protoplast fusion with intact cells, or polycations, e.g., polybrene, polyornithine, may also be used. For various techniques for transforming mammalian cells, see Keown et al., Methods in Enzymology, 185:527-537 (1990) and Mansour et al., Nature, 336:348-352 (1988).

Prokaryotic cells may be cultured in suitable culture media as described generally in Sambrook et al., supra. Examples of commercially available culture media include Ham's F10 (Sigma), Minimal Essential Medium (“MEM”, Sigma), RPMI-1640 (Sigma), and Dulbecco's Modified Eagle's Medium (“DMEM”, Sigma). Any such media may be supplemented as necessary with hormones and/or other growth factors (such as insulin, transferrin, or epidermal growth factor), salts (such as sodium chloride, calcium, magnesium, and phosphate), buffers (such as HEPES), nucleosides (such as adenosine and thymidine), antibiotics (such as Gentamycin™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

In general, principles, protocols, and practical techniques for maximizing the productivity of mammalian cell cultures can be found in Mammalian Cell Biotechnology: A Practical Approach, M. Butler, ed. (IRL Press, 1991).

The expressed polypeptides may be recovered from the culture medium as a secreted polypeptide, although may also be recovered from host cell lysates when directly produced without a secretory signal. If the polypeptide is membrane-bound, it can be released from the membrane using a suitable detergent solution (e.g. Triton-X 100) or its extracellular region may be released by enzymatic cleavage.

When the polypeptide is produced in a recombinant cell other than one of human origin, it is free of proteins or polypeptides of human origin. However, it is usually necessary to recover or purify the polypeptide from recombinant cell proteins or polypeptides to obtain preparations that are substantially homogeneous. As a first step, the culture medium or lysate may be centrifuged to remove particulate cell debris. The following are procedures exemplary of suitable purification procedures: by fractionation on an ion-exchange column; ethanol precipitation; reverse phase HPLC; chromatography on silica or on a cation-exchange resin such as DEAE; chromatofocusing; SDS-PAGE; ammonium sulfate precipitation; gel filtration using, for example, Sephadex G-75; and protein A Sepharose columns to remove contaminants such as IgG.

OPGL variants (or OPG variants or RANK variants) are contemplated for use in the invention. Such variants can be prepared using any suitable technique in the art. The variants can be prepared by introducing appropriate nucleotide changes into the ligand's (or receptor's) DNA, and/or by synthesis of the desired polypeptide. Those skilled in the art will appreciate that amino acid changes may alter post-translational processes of the ligand or receptor, such as changing the number or position of glycosylation sites or altering the membrane anchoring characteristics.

Variations in the native sequence or in various domains of the ligand (or receptor) described herein, can be made, for example, using any of the techniques and guidelines for conservative and non-conservative mutations set forth, for instance, in U.S. Pat. No. 5,364,934. Variations may be a substitution, deletion or insertion of one or more codons encoding the ligand or receptor that results in a change in the amino acid sequence of the ligand or receptor as compared with the respective native sequence (shown in the respective figures herein). Optionally the variation is by substitution of at least one amino acid with any other amino acid in one or more of the domains of the ligand or receptor. Guidance in determining which amino acid residue may be inserted, substituted or deleted without adversely affecting the desired activity may be found by comparing the sequence of the ligand or receptor with that of homologous known protein molecules and minimizing the number of amino acid sequence changes made in regions of high homology. Amino acid substitutions can be the result of replacing one amino acid with another amino acid having similar structural and/or chemical properties, such as the replacement of a leucine with a serine, i.e., conservative amino acid replacements. Insertions or deletions may optionally be in the range of about 1 to 5 amino acids. The variation allowed may be determined by systematically making insertions, deletions or substitutions of amino acids in the sequence and testing the resulting variants for activity exhibited by the full-length or mature native sequence.

OPGL polypeptide or receptor fragments are provided herein. Such fragments may be truncated at the N-terminus or C-terminus, or may lack internal residues, for example, when compared with a full-length native protein. Certain fragments lack amino acid residues that are not essential for a desired biological activity of the ligand or receptor polypeptide.

OPGL or receptor fragments may be prepared by any of a number of conventional techniques. Desired peptide fragments may be chemically synthesized. An alternative approach involves generating fragments by enzymatic digestion, e.g., by treating the protein with an enzyme known to cleave proteins at sites defined by particular amino acid residues, or by digesting the DNA with suitable restriction enzymes and isolating the desired fragment. Yet another suitable technique involves isolating and amplifying a DNA fragment encoding a desired polypeptide fragment, by polymerase chain reaction (PCR). Oligonucleotides that define the desired termini of the DNA fragment are employed at the 5′ and 3′ primers in the PCR.

In particular embodiments, conservative substitutions of interest are shown in Table 1 under the heading of preferred substitutions. If such substitutions result in a change in biological activity, then more substantial changes, denominated exemplary substitutions in Table 1, or as further described below in reference to amino acid classes, may be introduced and the products screened.

TABLE 1

| Original | Exemplary | Preferred |
|---|---|---|
| Residue | Substitutions | Substitutions |
| Ala (A) | val; leu; ile | val |
| Arg (R) | lys; gln; asn | lys |
| Asn (N) | gln; his; lys; arg | gln |
| Asp (D) | glu | glu |
| Cys (C) | ser | ser |
| Gln (Q) | asn | asn |
| Glu (E) | asp | asp |
| Gly (G) | pro; ala | ala |
| His (H) | asn; gln; lys; arg | arg |
| Ile (I) | leu; val; met; ala; phe; | leu |
| | norleucine | |
| Leu (L) | norleucine; ile; val; | ile |
| | met; ala; phe | |
| Lys (K) | arg; gln; asn | arg |
| Met (M) | leu; phe; ile | leu |
| Phe (F) | leu; val; ile; ala; tyr | leu |
| Pro (P) | ala | ala |
| Ser (S) | thr | thr |
| Thr (T) | ser | ser |
| Trp (W) | tyr; phe | tyr |
| Tyr (Y) | trp; phe; thr; ser | phe |
| Val (V) | ile; leu; met; phe; | leu |
| | ala; norleucine | |

Substantial modifications in function or immunological identity of the ligand or receptor polypeptide are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:
(1) hydrophobic: norleucine, met, ala, val, leu, ile;
(2) neutral hydrophilic: cys, ser, thr;
(3) acidic: asp, glu;
(4) basic: asn, gin, his, lys, arg;
(5) residues that influence chain orientation: gly, pro; and
(6) aromatic: trp, tyr, phe.

Non-conservative substitutions will entail exchanging a member of one of these classes for another class. Such substituted residues also may be introduced into the conservative substitution sites or, more preferably, into the remaining (non-conserved) sites.

The variations can be made using methods known in the art such as oligonucleotide-mediated (site-directed) mutagenesis, alanine scanning, and PCR mutagenesis. Site-directed mutagenesis (Carter et al., Nucl. Acids Res., 13:4331 (1986); Zoller et al., Nucl. Acids Res., 10:6487 (1987)], cassette mutagenesis [Wells et al., Gene, 34:315 (1985)], restriction selection mutagenesis [Wells et al., Philos. Trans. R. Soc. London SerA, 317:415 (1986)] or other known techniques can be performed on the cloned DNA to produce the variant DNA.

Scanning amino acid analysis can also be employed to identify one or more amino acids along a contiguous sequence. Among the preferred scanning amino acids are relatively small, neutral amino acids. Such amino acids include alanine, glycine, serine, and cysteine. Alanine is typically a preferred scanning amino acid among this group because it eliminates the side-chain beyond the beta-carbon and is less likely to alter the main-chain conformation of the variant (Cunningham and Wells, Science, 244: 1081-1085 (1989)]. Alanine is also typically preferred because it is the most common amino acid. Further, it is frequently found in both buried and exposed positions [Creighton, The Proteins, (W.H. Freeman & Co., N.Y.); Chothia, J. Mol. Biol., 150:1 (1976)]. If alanine substitution does not yield adequate amounts of variant, an isoteric amino acid can be used.

Soluble forms of OPGL or receptors may also be employed in the methods of the invention. Such soluble forms of OPGL or receptors may comprise or consist of extracellular domains of the respective ligand or receptor (and lacking transmembrane and intracellular domains). The extracellular domain sequences themselves may be used, or may be further modified as described below (such as by fusing to an immunoglobulin, epitope tag or leucine zipper). Certain extracellular domain regions of OPGL, OPG and RANK have been described in the literature and may be further delineated using techniques known to the skilled artisan. Optionally, OPG ligand contemplated for use in the methods includes a polypeptide having the contiguous sequence of amino acid residues 70 to 317 or 75 to 316 of FIG. 1B (SEQ ID NO:1). Optionally, OPG receptor contemplated for use in the methods includes a polypeptide having the contiguous sequence of amino acid residues 22 to 401 of FIG. 2B (SEQ ID NO:3). Optionally, RANK receptor contemplated for use in the methods includes a polypeptide having the contiguous sequence of amino acid residues 29 to 212 of FIG. 3B (SEQ ID NO:5). Those skilled in the art will be able to select, without undue experimentation, a desired extracellular domain sequence to employ. An example of such an extracellular domain sequence of OPGL having the desired biological activity is described in the Examples section below.

In another embodiment, the OPGL or receptor may be covalently modified by linking the polypeptide to one of a variety of nonproteinaceous polymers, e.g., polyethylene glycol (PEG), polypropylene glycol, or polyoxyalkylenes, in the manner set forth in U.S. Pat. Nos. 4,640,835; 4,496,689; 4,301,144; 4,670,417; 4,791,192 or 4,179,337. Such pegylated forms of the polypeptide may be prepared using techniques known in the art. Optionally, the OPGL or receptor may be covalently modified by linking the polypeptide to one or more polyglutamate molecules.

Leucine zipper forms of these molecules are also contemplated by the invention. “Leucine zipper” is a term in the art used to refer to a leucine rich sequence that enhances, promotes, or drives dimerization or trimerization of its fusion partner (e.g., the sequence or molecule to which the leucine zipper is fused or linked to). Various leucine zipper polypeptides have been described in the art. See, e.g., Landschulz et al., Science, 240:1759 (1988); U.S. Pat. No. 5,716,805; WO 94/10308; Hoppe et al., FEBS Letters, 344:1991 (1994); Maniatis et al., Nature, 341:24 (1989). Those skilled in the art will appreciate that a leucine zipper sequence may be fused at either the 5′ or 3′ end of the polypeptide molecule.

The OPGL or receptor polypeptides of the present invention may also be modified in a way to form chimeric molecules by fusing the polypeptide to another, heterologous polypeptide or amino acid sequence. Preferably, such heterologous polypeptide or amino acid sequence is one which acts to oligimerize the chimeric molecule. In one embodiment, such a chimeric molecule comprises a fusion of the OPGL polypeptide with a tag polypeptide which provides an epitope to which an anti-tag antibody can selectively bind. The epitope tag is generally placed at the amino- or carboxyl-terminus of the receptor polypeptide. The presence of such epitope-tagged forms of the receptor can be detected using an antibody against the tag polypeptide. Also, provision of the epitope tag enables the receptor to be readily purified by affinity purification using an anti-tag antibody or another type of affinity matrix that binds to the epitope tag. Various tag polypeptides and their respective antibodies are well known in the art. Examples include poly-histidine (poly-his) or poly-histidine-glycine (poly-his-gly) tags; the flu HA tag polypeptide and its antibody 12CA5 [Field et al., Mol. Cell. Biol., 8:2159-2165 (1988); the c-myc tag and the 8F9, 3C7, 6E10, G4, B7 and 9E10 antibodies thereto [Evan et al., Molecular and Cellular Biology, 5:3610-3616 (1985)]; and the Herpes Simplex virus glycoprotein D (gD) tag and its antibody [Paborsky et al., Protein Engineering, 3(6):547-553 (1990)]. Other tag polypeptides include the Flag-peptide [Hopp et al., BioTechnology, 6:1204-1210 (1988)]; the KT3 epitope peptide [Martin et al., Science, 255:192-194 (1992)]; an α-tubulin epitope peptide [Skinner et al., J. Biol. Chem., 266:15163-15166 (1991)]; and the T7 gene 10 protein peptide tag (Lutz-Freyermuth et al., Proc. Natl. Acad. Sci. USA, 87:6393-6397 (1990)].

Immunoadhesin molecules are further contemplated for use in the methods herein. Receptor immunoadhesins may comprise various forms of OPG receptor or RANK receptor, such as the full length polypeptide as well as soluble forms of the receptor which comprise an extracellular domain (ECD) sequence or a fragment of the ECD sequence. In one embodiment, the molecule may comprise a fusion of the OPG receptor or RANK receptor with an immunoglobulin or a particular region of an immunoglobulin. For a bivalent form of the immunoadhesin, such a fusion could be to the Fc region of an IgG molecule. The Ig fusions preferably include the substitution of a soluble (transmembrane domain deleted or inactivated) form of the receptor polypeptide in place of at least one variable region within an Ig molecule. In a particularly preferred embodiment, the immunoglobulin fusion includes the hinge, CH2 and CH3, or the hinge, CH1, CH2 and CH3 regions of an IgG1 molecule. For the production of immunoglobulin fusions, see also U.S. Pat. No. 5,428,130 issued Jun. 27, 1995 and Chamow et al., TIBTECH, 14:52-60 (1996).

Optionally, the immunoadhesin combines the binding domain(s) of the adhesin (e.g. the extracellular domain (ECD) of a receptor) with the Fc region of an immunoglobulin heavy chain. Ordinarily, when preparing the immunoadhesins of the present invention, nucleic acid encoding the binding domain of the adhesin will be fused C-terminally to nucleic acid encoding the N-terminus of an immunoglobulin constant domain sequence, however N-terminal fusions are also possible.

Typically, in such fusions the encoded chimeric polypeptide will retain at least functionally active hinge, C_{H}2 and C_{H}3 domains of the constant region of an immunoglobulin heavy chain. Fusions are also made to the C-terminus of the Fc portion of a constant domain, or immediately N-terminal to the C_{H}1 of the heavy chain or the corresponding region of the light chain. The precise site at which the fusion is made is not critical; particular sites are well known and may be selected in order to optimize the biological activity, secretion, or binding characteristics of the immunoadhesin.

In a preferred embodiment, the adhesin sequence is fused to the N-terminus of the Fc region of immunoglobulin G₁ (IgG₁). It is possible to fuse the entire heavy chain constant region to the adhesin sequence. However, more preferably, a sequence beginning in the hinge region just upstream of the papain cleavage site which defines IgG Fc chemically (i.e. residue 216, taking the first residue of heavy chain constant region to be 114), or analogous sites of other immunoglobulins is used in the fusion. In a particularly preferred embodiment, the adhesin amino acid sequence is fused to (a) the hinge region and C_{H}2 and C_{H}3 or (b) the C_{H}1, hinge, C_{H}2 and C_{H}3 domains, of an IgG heavy chain.

For bispecific immunoadhesins, the immunoadhesins are assembled as multimers, and particularly as heterodimers or heterotetramers. Generally, these assembled immunoglobulins will have known unit structures. A basic four chain structural unit is the form in which IgG, IgD, and IgE exist. A four chain unit is repeated in the higher molecular weight immunoglobulins; IgM generally exists as a pentamer of four basic units held together by disulfide bonds. IgA globulin, and occasionally IgG globulin, may also exist in multimeric form in serum. In the case of multimer, each of the four units may be the same or different.

Various exemplary assembled immunoadhesins within the scope herein are schematically diagrammed below:
(a) AC_{L}-AC_{L};
(b) AC_{H}-(AC_{H}, AC_{L}-AC_{H}, AC_{L}-V_{H}C_{H}, or V_{L}C_{L}-AC_{H});
(c) AC_{L}-AC_{H}-(AC_{L}-AC_{H}, AC_{L}-V_{H}C_{H}, V_{L}C_{L}-AC_{H}, or V_{L}C_{L}-V_{H}C_{H})
(d) AC_{L}-V_{H}C_{H}-(AC_{H}, or AC_{L}-V_{H}C_{H}, or V_{L}C_{L}-AC_{H});
(e) V_{L}C_{L}-AC_{H}-(AC_{L}-V_{H}C_{H}, or V_{L}C_{L}-AC_{H}); and
(f) (A-Y)ₙ-(V_{L}C_{L}-V_{H}C_{H})₂,
   wherein each A represents identical or different adhesin amino acid sequences;
V_{L} is an immunoglobulin light chain variable domain;
V_{H} is an immunoglobulin heavy chain variable domain;
C_{L} is an immunoglobulin light chain constant domain;
C_{H} is an immunoglobulin heavy chain constant domain;
n is an integer greater than 1;
Y designates the residue of a covalent cross-linking agent.

In the interests of brevity, the foregoing structures only show key features; they do not indicate joining (J) or other domains of the immunoglobulins, nor are disulfide bonds shown. However, where such domains are required for binding activity, they shall be constructed to be present in the ordinary locations which they occupy in the immunoglobulin molecules.

Alternatively, the adhesin sequences can be inserted between immunoglobulin heavy chain and light chain sequences, such that an immunoglobulin comprising a chimeric heavy chain is obtained. In this embodiment, the adhesin sequences are fused to the 3′ end of an immunoglobulin heavy chain in each arm of an immunoglobulin, either between the hinge and the C_{H}2 domain, or between the C_{H}2 and C_{H}3 domains. Similar constructs have been reported by Hoogenboom et al., Mol. Immunol., 28:1027-1037 (1991).

Although the presence of an immunoglobulin light chain is not required in the immunoadhesins of the present invention, an immunoglobulin light chain might be present either covalently associated to an adhesin-immunoglobulin heavy chain fusion polypeptide, or directly fused to the adhesin. In the former case, DNA encoding an immunoglobulin light chain is typically coexpressed with the DNA encoding the adhesin-immunoglobulin heavy chain fusion protein. Upon secretion, the hybrid heavy chain and the light chain will be covalently associated to provide an immunoglobulin-like structure comprising two disulfide-linked immunoglobulin heavy chain-light chain pairs. Methods suitable for the preparation of such structures are, for example, disclosed in U.S. Pat. No. 4,816,567, issued 28 Mar. 1989.

Immunoadhesins are most conveniently constructed by fusing the cDNA sequence encoding the adhesin portion in-frame to an immunoglobulin cDNA sequence. However, fusion to genomic immunoglobulin fragments can also be used (see, e.g. Aruffo et al., Cell, 61:1303-1313 (1990); and Stamenkovic et al., Cell, 66:1133-1144 (1991)). The latter type of fusion requires the presence of Ig regulatory sequences for expression. cDNAs encoding IgG heavy-chain constant regions can be isolated based on published sequences from cDNA libraries derived from spleen or peripheral blood lymphocytes, by hybridization or by polymerase chain reaction (PCR) techniques. The cDNAs encoding the “adhesin” and the immunoglobulin parts of the immunoadhesin are inserted in tandem into a plasmid vector that directs efficient expression in the chosen host cells.

Examples of such soluble ECD sequences include polypeptides comprising amino acids 22 to 401 of the OPG receptor sequence shown in FIG. 2B. The OPG receptor receptor immunoadhesin can be made according to any of the methods described in the art.

RANK receptor immunoadhesins can be similarly constructed. Examples of soluble ECD sequences for use in constructing RANK receptor immunoadhesins may include polypeptides comprising amino acids 29 to 212 of the RANK sequence shown in FIG. 3B.

It is contemplated that anti-OPGL antibodies, anti-OPG receptor antibodies, or anti-RANK receptor antibodies may also be employed in the presently disclosed methods. Examples of such molecules include neutralizing or blocking antibodies which can preferably inhibit binding of OPGL to the OPG or to the RANK receptors. The anti-OPGL antibodies, anti-OPG, or anti-RANK antibodies may be monoclonal antibodies. Monoclonal antibodies may be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro.

The immunizing agent will typically include the OPG or RANK polypeptide, or OPGL polypeptide, or a fusion protein thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell [Goding, Monoclonal Antibodies: Principles and Practice, Academic Press, (1986) pp. 59-103]. Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies [Kozbor, J. Immunol., 133:3001 (1984); Brodeur et al., Monoclonal Antibody Production Techniques and Applications, Marcel Dekker, Inc., New York, (1987) pp. 51-63].

The culture medium in which the hybridoma cells are cultured can then be assayed for the presence of monoclonal antibodies directed against OPGL, OPG or RANK. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). Optionally, the anti-OPGL, anti-OPG, or anti-RANK antibodies will have a binding affinity of at least 10 nM, preferably, of at least 5 nM, and more preferably, of at least 1nM for the respective receptor or ligand, as determined in a binding assay.

After the desired hybridoma cells are identified, the clones may be subcloned by limiting dilution procedures and grown by standard methods [Goding, supra]. Suitable culture media for this purpose include, for example, Dulbecco's Modified Eagle's Medium and RPMI-1640 medium. Alternatively, the hybridoma cells may be grown in vivo as ascites in a mammal.

The monoclonal antibodies secreted by the subclones may be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies may also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA may be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences [U.S. Pat. No. 4,816,567; Morrison et al., supra] or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

The antibodies may be monovalent antibodies. Methods for preparing monovalent antibodies are well known in the art. For example, one method involves recombinant expression of immunoglobulin light chain and modified heavy chain. The heavy chain is truncated generally at any point in the Fc region so as to prevent heavy chain crosslinking. Alternatively, the relevant cysteine residues are substituted with another amino acid residue or are deleted so as to prevent crosslinking.

In vitro methods are also suitable for preparing monovalent antibodies. Digestion of antibodies to produce fragments thereof, particularly, Fab fragments, can be accomplished using routine techniques known in the art.

In a further embodiment, antibodies or antibody fragments can be isolated from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991) describe the isolation of murine and human antibodies, respectively, using phage libraries. Subsequent publications describe the production of high affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al., Nuc. Acids. Res., 21:2265-2266 (1993)). Thus, these techniques are viable alternatives to traditional monoclonal antibody hybridoma techniques for isolation of monoclonal antibodies.

The DNA also may be modified, for example, by substituting the coding sequence for human heavy- and light-chain constant domains in place of the homologous murine sequences (U.S. Pat. No. 4,816,567; Morrison, et al., Proc. Natl Acad. Sci. USA, 81:6851 (1984)), or by covalently joining to the immunoglobulin coding sequence all or part of the coding sequence for a non-immunoglobulin polypeptide.

Typically such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

A humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody (Sims et al., J. Immunol., 151:2296 (1993); Chothia et al., J. Mol. Biol., 196:901 (1987)). Another method uses a particular framework derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immnol., 151:2623 (1993)).

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the CDR residues are directly and most substantially involved in influencing antigen binding.

Alternatively, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (J_{H}) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggermann et al., Year in Immuno., 7:33 (1993); and Duchosal et al. , Nature, 355:258 (1992). Human antibodies can also be derived from phage-display libraries (Hoogenboom et al., J. Mol. Biol., 227:381 (1991); Marks et al., J. Mol. Biol., 222:581-597 (1991); Vaughan et al., Nature Biotech, 14:309 (1996)).

B. Formulations

The OPGL polypeptides (or agonist or antagonist) described herein are preferably employed in a carrier. Suitable carriers and their formulations are described in Remington's Pharmaceutical Sciences, 16th ed., 1980, Mack Publishing Co., edited by Osol et al. Typically, an appropriate amount of a pharmaceutically-acceptable salt is used in the carrier to render the formulation isotonic. Examples of the carrier include saline, Ringer's solution and dextrose solution. The pH of the solution is preferably from about 5 to about 8, and more preferably from about 7.4 to about 7.8. It will be apparent to those persons skilled in the art that certain carriers may be more preferable depending upon, for instance, the route of administration and concentration of agent being administered. The carrier may be in the form of a lyophilized formulation or aqueous solution.

Acceptable carriers, excipients, or stabilizers are preferably nontoxic to cells and/or recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; and/or non-ionic surfactants such as TWEEN™, PLURONICS™ or polyethylene glycol (PEG).

The OPGL (or agonist or antagonist) may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

The formulation herein may also contain more than one active compound as necessary for the particular indication being treated, preferably those with complementary activities that do not adversely affect each other. Hence, the present application contemplates combining the OPGL (or agonist or antagonist) with one or more other therapeutic agent(s), which depend on the particular indication being treated. While the agent may be an endocrine agent such as a GH, a GHRP, a GHRH, a GH secretagogue, an IGFBP, ALS, a GH complexed with a GHBP, it may optionally be a cytotoxic agent. For instance, the OPGL (or agonist or antagonist) may be co-administered with another peptide (or multivalent antibodies), a monovalent or bivalent antibody (or antibodies), chemotherapeutic agent(s) (including cocktails of chemotherapeutic agents), other cytotoxic agent(s), anti-angiogenic agent(s), cytokines, and/or growth inhibitory agent(s). Where the agent induces apoptosis, it may be particularly desirable to combine the peptide with one or more other therapeutic agent(s) that also induce apoptosis. For instance, it may be combined with pro-apoptotic antibodies (e.g. bivalent or multivalent antibodies) directed against B-cell surface antigens (e.g. RITUXAN®, ZEVALIN® or BEXXAR® anti-CD20 antibodies) and/or with (1) pro-apoptotic antibodies (e.g. bivalent or multivalent antibodies directed against a receptor in the TNF receptor superfamily, such as anti-DR4 or anti-DR5 antibodies) or (2) cytokines in the TNF family of cytokines (e.g. Apo2L). Likewise, it may be administered along with anti-ErbB antibodies (e.g. HERCEPTIN® anti-HER2 antibody) alone or combined with (1) and/or (2). Alternatively, or additionally, the patient may receive combined radiation therapy (e.g. external beam irradiation or therapy with a radioactive labeled agent, such as an antibody), ovarian ablation, chemical or surgical, or high-dose chemotherapy along with bone marrow transplantation or peripheral-blood stem-cell rescue or transplantation. Such combined therapies noted above include combined administration (where the two or more agents are included in the same or separate formulations), and separate administration, in which case, administration of the OPGL (or agonist or antagonist) can occur prior to, and/or following, administration of the adjunct therapy or therapies. The effective amount of such other agents depends on the amount of OPGL (or agonist or antagonist) present in the formulation, the type of disorder or treatment, and other factors discussed above. These are generally used in the same dosages and with administration routes as used hereinbefore or about from 1 to 99% of the heretofore employed dosages.

The formulations to be used for in vivo administration should be sterile. This is readily accomplished by filtration through sterile filtration membranes.

Sustained-release preparations may be prepared. Suitable examples of sustained-release preparations include semipermeable matrices of solid hydrophobic polymers, which matrices are in the form of shaped articles, e.g. films, or microcapsules. Examples of sustained-release matrices include polyesters, hydrogels (for example,poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

C. Modes of Therspy

The OPGL (or agonist or antagonist) molecules described herein are useful in treating various pathological conditions, such as immune related diseases. Certain of these conditions can be treated by stimulating monocyte secretion of one or more cytokines or chemokines in a mammal through administration of the OPGL or agonist molecule described herein. Other types of immune related conditions can be treated using the antagonist molecules described herein to inhibit or neutralize monocyte secretion of such cytokines or chemokines.

Diagnosis in mammals of the various pathological conditions described herein can be made by the skilled practitioner. Diagnostic techniques are available in the art which allow, e.g., for the diagnosis or detection of immune related disease in a mammal. In systemic lupus erythematosus, the central mediator of disease is the production of auto-reactive antibodies to self proteins/tissues and the subsequent generation of immune-mediated inflammation. Multiple organs and systems are affected clinically including kidney, lung, musculoskeletal system, mucocutaneous, eye, central nervous system, cardiovascular system, gastrointestinal tract, bone marrow and blood.

Rheumatoid arthritis (RA) is a chronic systemic autoimmune inflammatory disease that mainly involves the synovial membrane of multiple joints with resultant injury to the articular cartilage. The pathogenesis is T lymphocyte dependent and is associated with the production of rheumatoid factors, auto-antibodies directed against self IgG, with the resultant formation of immune complexes that attain high levels in joint fluid and blood. These complexes in the joint may induce the marked infiltrate of lymphocytes and monocytes into the synovium and subsequent marked synovial changes; the joint space/fluid if infiltrated by similar cells with the addition of numerous neutrophils. Tissues affected are primarily the joints, often in symmetrical pattern. However, extra-articular disease also occurs in two major forms. One form is the development of extra-articular lesions with ongoing progressive joint disease and typical lesions of pulmonary fibrosis, vasculitis, and cutaneous ulcers. The second form of extra-articular disease is the so called Felty's syndrome which occurs late in the RA disease course, sometimes after joint disease has become quiescent, and involves the presence of neutropenia, thrombocytopenia and splenomegaly. This can be accompanied by vasculitis in multiple organs with formations of infarcts, skin ulcers and gangrene. Patients often also develop rheumatoid nodules in the subcutis tissue overlying affected joints; the nodules late stage have necrotic centers surrounded by a mixed inflammatory cell infiltrate. Other manifestations which can occur in RA include: pericarditis, pleuritis, coronary arteritis, intestitial pneumonitis with pulmonary fibrosis, keratoconjunctivitis sicca, and rhematoid nodules.

Juvenile chronic arthritis is a chronic idiopathic inflammatory disease which begins often at less than 16 years of age. Its phenotype has some similarities to RA; some patients which are rhematoid factor positive are classified as juvenile rheumatoid arthritis. The disease is sub-classified into three major categories: pauciarticular, polyarticular, and systemic. The arthritis can be severe and is typically destructive and leads to joint ankylosis and retarded growth. Other manifestations can include chronic anterior uveitis and systemic amyloidosis.

Spondyloarthropathies are a group of disorders with some common clinical features and the common association with the expression of HLA-B27 gene product. The disorders include: ankylosing sponylitis, Reiter's syndrome (reactive arthritis), arthritis associated with inflammatory bowel disease, spondylitis associated with psoriasis, juvenile onset spondyloarthropathy and undifferentiated spondyloarthropathy. Distinguishing features include sacroileitis with or without spondylitis; inflammatory asymmetric arthritis; association with HLA-B27 (a serologically defined allele of the HLA-B locus of class I MHC); ocular inflammation, and absence of autoantibodies associated with other rheumatoid disease. The cell most implicated as key to induction of the disease is the CD8+T lymphocyte, a cell which targets antigen presented by class I MHC molecules. CD8+ T cells may react against the class I MHC allele HLA-B27 as if it were a foreign peptide expressed by MHC class I molecules. It has been hypothesized that an epitope of HLA-B27 may mimic a bacterial or other microbial antigenic epitope and thus induce a CD8+ T cells response.

Systemic sclerosis (scleroderma) has an unknown etiology. A hallmark of the disease is induration of the skin; likely this is induced by an active inflammatory process. Scleroderma can be localized or systemic; vascular lesions are common and endothelial cell injury in the microvasculature is an early and important event in the development of systemic sclerosis; the vascular injury may be immune mediated. An immunologic basis is implied by the presence of mononuclear cell infiltrates in the cutaneous lesions and the presence of anti-nuclear antibodies in many patients. ICAM-1 is often upregulated on the cell surface of fibroblasts in skin lesions suggesting that T cell interaction with these cells may have a role in the pathogenesis of the disease. Other organs involved include: the gastrointestinal tract: smooth muscle atrophy and fibrosis resulting in abnormal peristalsis/motility; kidney: concentric subendothelial intimal proliferation affecting small arcuate and interlobular arteries with resultant reduced renal cortical blood flow, results in proteinuria, azotemia and hypertension; skeletal muscle: atrophy, interstitial fibrosis; inflammation; lung: interstitial pneumonitis and interstitial fibrosis; and heart: contraction band necrosis, scarring/fibrosis.

Idiopathic inflammatory myopathies including dermatomyositis, polymyositis and others are disorders of chronic muscle inflammation of unknown etiology resulting in muscle weakness. Muscle injury/inflammation is often symmetric and progressive. Autoantibodies are associated with most forms. These myositis-specific autoantibodies are directed against and inhibit the function of components, proteins and RNA's, involved in protein synthesis.

Sjogren's syndrome is due to immune-mediated inflammation and subsequent functional destruction of the tear glands and salivary glands. The disease can be associated with or accompanied by inflammatory connective tissue diseases. The disease is associated with autoantibody production against Ro and La antigens, both of which are small RNA-protein complexes. Lesions result in keratoconjunctivitis sicca, xerostomia, with other manifestations or associations including bilary cirrhosis, peripheral or sensory neuropathy, and palpable purpura.

Systemic vasculitis are diseases in which the primary lesion is inflammation and subsequent damage to blood vessels which results in ischemia/necrosis/degeneration to tissues supplied by the affected vessels and eventual end-organ dysfunction in some cases. Vasculitides can also occur as a secondary lesion or sequelae to other immune-inflammatory mediated diseases such as rheumatoid arthritis, systemic sclerosis, etc., particularly in diseases also associated with the formation of immune complexes. Diseases in the primary systemic vasculitis group include: systemic necrotizing vasculitis: polyarteritis nodosa, allergic angiitis and granulomatosis, polyangiitis; Wegener's granulomatosis; lymphomatoid granulomatosis; and giant cell arteritis. Miscellaneous vasculitides include: mucocutaneous lymph node syndrome (MLNS or Kawasaki's disease), isolated CNS vasculitis, Behet's disease, thromboangiitis obliterans (Buerger's disease) and cutaneous necrotizing venulitis. The pathogenic mechanism of most of the types of vasculitis listed is believed to be primarily due to the deposition of immunoglobulin complexes in the vessel wall and subsequent induction of an inflammatory response either via ADCC, complement activation, or both.

Sarcoidosis is a condition of unknown etiology which is characterized by the presence of epithelioid granulomas in nearly any tissue in the body; involvement of the lung is most common. The pathogenesis involves the persistence of activated macrophages and lymphoid cells at sites of the disease with subsequent chronic sequelae resultant from the release of locally and systemically active products released by these cell types.

Autoimmune hemolytic anemia including autoimmune hemolytic anemia, immune pancytopenia, and paroxysmal noctural hemoglobinuria is a result of production of antibodies that react with antigens expressed on the surface of red blood cells (and in some cases other blood cells including platelets as well) and is a reflection of the removal of those antibody coated cells via complement mediated lysis and/or ADCC/Fc-receptor-mediated mechanisms.

In autoimmune thrombocytopenia including thrombocytopenic purpura, and immune-mediated thrombocytopenia in other clinical settings, platelet destruction/removal occurs as a result of either antibody or complement attaching to platelets and subsequent removal by complement lysis, ADCC or FC-receptor mediated mechanisms.

Thyroiditis including Grave's disease, Hashimoto's thyroiditis, juvenile lymphocytic thyroiditis, and atrophic thyroiditis, are the result of an autoimmune response against thyroid antigens with production of antibodies that react with proteins present in and often specific for the thyroid gland. Experimental models exist including spontaneous models: rats (BUF and BB rats) and chickens (obese chicken strain); inducible models: immunization of animals with either thyroglobulin, thyroid microsomal antigen (thyroid peroxidase).

Type I diabetes mellitus or insulin-dependent diabetes is the autoimmune destruction of pancreatic islet β cells; this destruction is mediated by auto-antibodies and auto-reactive T cells. Antibodies to insulin or the insulin receptor can also produce the phenotype of insulin-non-responsiveness.

Immune mediated renal diseases, including glomerulonephritis and tubulointerstitial nephritis, are the result of antibody or T lymphocyte mediated injury to renal tissue either directly as a result of the production of autoreactive antibodies or T cells against renal antigens or indirectly as a result of the deposition of antibodies and/or immune complexes in the kidney that are reactive against other, non-renal antigens. Thus other immune-mediated diseases that result in the formation of immune-complexes can also induce immune mediated renal disease as an indirect sequelae. Both direct and indirect immune mechanisms result in inflammatory response that produces/induces lesion development in renal tissues with resultant organ function impairment and in some cases progression to renal failure. Both humoral and cellular immune mechanisms can be involved in the pathogenesis of lesions.

Demyelinating diseases of the central and peripheral nervous systems, including Multiple Sclerosis; idiopathic demyelinating polyneuropathy or Guillain-Barr syndrome; and Chronic Inflammatory Demyelinating Polyneuropathy, are believed to have an autoimmune basis and result in nerve demyelination as a result of damage caused to oligodendrocytes or to myelin directly. In MS there is evidence to suggest that disease induction and progression is dependent on T lymphocytes. Multiple Sclerosis is a demyelinating disease that is T lymphocyte-dependent and has either a relapsing-remitting course or a chronic progressive course. The etiology is unknown; however, viral infections, genetic predisposition, environment, and autoimmunity all contribute. Lesions contain infiltrates of predominantly T lymphocyte mediated, microglial cells and infiltrating macrophages; CD4+T lymphocytes are the predominant cell type at lesions. The mechanism of oligodendrocyte cell death and subsequent demyelination is not known but is likely T lymphocyte driven.

Inflammatory and Fibrotic Lung Disease, including Eosinophilic Pneumonias; Idiopathic Pulmonary Fibrosis, and Hypersensitivity Pneumonitis may involve a disregulated immune-inflammatory response. Inhibition of that response would be of therapeutic benefit.

Autoimmune or Immune-mediated Skin Disease including Bullous Skin Diseases, Erythema Multiforme, and Contact Dermatitis are mediated by auto-antibodies, the genesis of which is T lymphocyte-dependent.

Psoriasis is a T lymphocyte-mediated inflammatory disease. Lesions contain infiltrates of T lymphocytes, macrophages and antigen processing cells, and some neutrophils.

Allergic diseases, including asthma; allergic rhinitis; atopic dermatitis; food hypersensitivity; and urticaria are T lymphocyte dependent. These diseases are predominantly mediated by T lymphocyte induced inflammation, IgE mediated-inflammation or a combination of both.

Transplantation associated diseases, including Graft rejection and Graft-Versus-Host-Disease (GVHD) are T lymphocyte-dependent; inhibition of T lymphocyte function is ameliorative.

Other diseases in which intervention of the immune and/or inflammatory response have benefit are Infectious disease including but not limited to viral infection (including but not limited to AIDS, hepatitis A, B, C, D, E) bacterial infection, fungal infections, and protozoal and parasitic infections (molecules (or derivatives/agonists) which stimulate the MLR can be utilized therapeutically to enhance the immune response to infectious agents), diseases of immunodeficiency (molecules/derivatives/agonists) which stimulate the MLR can be utilized therapeutically to enhance the immune response for conditions of inherited, acquired, infectious induced (as in HIV infection), or iatrogenic (i.e. as from chemotherapy) immunodeficiency), and neoplasia.

The OPGL (or agonist or antagonist) can be administered in accord with known methods, such as intravenous administration as a bolus or by continuous infusion over a period of time, by intramuscular, intraperitoneal, intracerebrospinal, subcutaneous, intra-articular, intrasynovial, intrathecal, oral, topical, or inhalation routes. Optionally, administration may be performed through mini-pump infusion using various commercially available devices. The OPGL (or agonist or antagonist) may also be employed using gene therapy techniques which have been described in the art.

Effective dosages and schedules for administering OPGL (or agonist or antagonist) may be determined empirically, and making such determinations is within the skill in the art. Single or multiple dosages may be employed. It is presently believed that an effective dosage or amount of OPGL, for example, used alone may range from about 1 μg/kg to about 100 mg/kg of body weight or more per day. Interspecies scaling of dosages can be performed in a manner known in the art, e.g., as disclosed in Mordenti et al., Pharmaceut. Res., 8:1351 (1991). When in vivo administration of OPGL is employed, normal dosage amounts may vary from about 10 ng/kg to up to 100 mg/kg of mammal body weight or more per day, preferably about 1 μg/kg/day to 10 mg/kg/day, depending upon the route of administration. Guidance as to particular dosages and methods of delivery is provided in the literature; see, for example, U.S. Pat. Nos. 4,657,760; 5,206,344; or 5,225,212. It is anticipated that different formulations will be effective for different treatment compounds and different disorders, that administration targeting one organ or tissue, for example, may necessitate delivery in a manner different from that to another organ or tissue. Those skilled in the art will understand that the dosage of OPGL (or agonist or antagonist molecule) that must be administered will vary depending on, for example, the mammal which will receive the therapy, the route of administration, and other drugs or therapies being administered to the mammal. It is contemplated that combinations of any one or more of the agonists or antagonists disclosed herein may also be employed in the methods described by the present invention.

It is contemplated that yet additional therapies may be employed in the methods. The one or more other therapies may include but are not limited to, administration of radiation therapy, cytokine(s), growth inhibitory agent(s), chemotherapeutic agent(s), cytotoxic agent(s), tyrosine kinase inhibitors, ras farnesyl transferase inhibitors, angiogenesis inhibitors, and cyclin-dependent kinase inhibitors which are known in the art and defined further with particularity in Section I above. Further therapies include but are not limited to blocking antibodies or immunoadhesin molecules which neutralize the activity of various TNF family molecules, such as neutralizing antibodies of TNF-alpha (i.e., Remicade™), CD40 Ligand/CD40 receptor, or OX40 ligand/OX40 receptor, or receptor-immunoglobulin constructs such as Embrel™.

The OPGL (or agonist or antagonist) and one or more other therapies may be administered concurrently or sequentially. Following administration of such therapy, treated cells in vitro can be analyzed. Where there has been in vivo treatment, a treated mammal can be monitored in various ways well known to the skilled practitioner.

D. Articles of Manufacture

In another embodiment of the invention, articles of manufacture containing materials useful for the treatment of the disorders described above are provided. The article of manufacture comprises a container and a label. Suitable containers include, for example, bottles, vials, syringes, and test tubes. The containers may be formed from a variety of materials such as glass or plastic. The container holds a composition which is effective for treating the condition and may have a sterile access port (for example the container may be an intravenous solution bag or a vial having a stopper pierceable by a hypodermic injection needle). The active agents in the composition may comprise OPGL or agonists or antagonists, as described herein. The label on, or associated with, the container indicates that the composition is used for treating the condition of choice. The article of manufacture may further comprise a second container comprising a pharmaceutically-acceptable carrier, such as phosphate-buffered saline, Ringer's solution and dextrose solution. It may further include other materials desirable from a commercial and user standpoint, including other buffers, diluents, filters, needles, syringes, and package inserts with instructions for use.

The following examples are offered by way of illustration and not by way of limitation. The disclosures of all citations in the specification are expressly incorporated herein by reference.

### EXAMPLE 1

### Proliferation of PBMC by OPG Ligand in Vitro

An in vitro assay was conducted to examine the effects of OPG ligand on human peripheral blood mononuclear cells (PBMC).

Human blood was purified over LSM (ICN Pharmaceutical, Inc.), washed 2× with PBS, resuspended into complete medium (RPMI 1640 containing 10% FBS heat-inactivated and 50 U/ml penicillin, 50 ug/ml streptomycin) and plated at 37° C. for 30 minutes at 5×10⁷ cells/150 mm tissue culture plate. Non-adherent cells were re-plated for another 30 minutes under the same conditions. Adherent cells were harvested gently using a cell-scraper and adjusted to either 3×10⁶/ml or 5×10⁶/ml. Enriched monocytes were plated-out at either 3×10⁶/well or 5×10⁵/well in 96-well flat-bottom tissue-culture plates.

Monocytes were cultured in the 96-well flat-bottom plates in the presence of serially-diluted recombinant soluble human OPGL Flag-tagged molecule with media and Pokeweed mitogen (PWM) (5 ug/ml) (Sigma) and/or LPS (100 ng/ml) (Sigma) as negative and positive controls, respectively, at 37° C., 5% CO₂. The OPG ligand was a recombinant soluble, Flag-tagged OPG ligand (comprising amino acids 75-316 of the extracellular domain of human OPGL; see FIG. 1, SEQ ID NO:1) purchased from Alexis Corporation. Proliferation of human PBMC was measured by pulsing the cultures with ³H-Thymidine for the last 16 hours of the culture. After 4 days, plates were spun briefly and supernatants were collected. Thymidine incorporation was measured by scintillation counting.

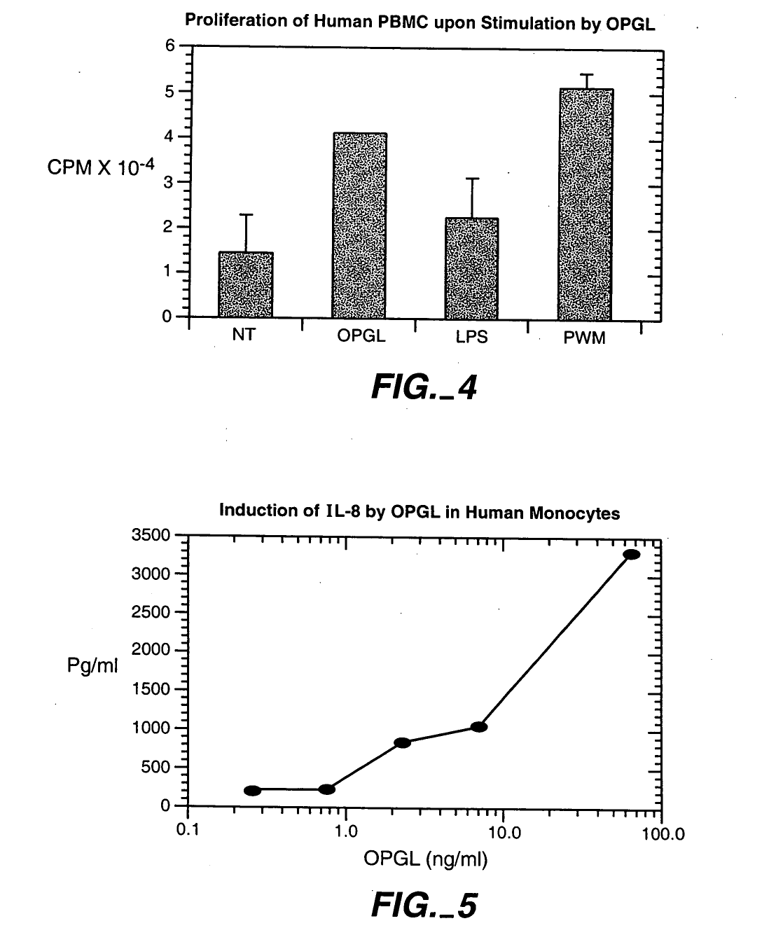

The results are shown in FIG. 4, and the proliferation of cells is reported as CPM×10⁻⁴.

### EXAMPLE 2

### Induction of IL-8 by OPG Ligand

An in vitro assay was conducted to examine the effects of OPG ligand on IL-8 induction in human monocytes. The assay was conducted essentially as described in Example 1 except that the plates were spun briefly and supernatants were collected after a 24 hour incubation. The varying concentration of soluble OPGL added to the cultures is shown in FIG. 5. No radioisotope was added to the culture plates. The supernatants were then measured by ELISA (Endogen) for IL-8 levels, as per manufacturer's recommendation.

The results are shown in FIG. 5, and indicate the levels of IL-8 as Pg/ml.

### EXAMPLE 3

### Induction of TNF-alpha by OPG Ligand

An in vitro assay was conducted to examine the effects of OPG ligand on TNF-alpha induction in human monocytes. The assay was conducted essentially as described in Example 2. The supernatants were then measured by ELISA (Endogen) for TNF-alpha levels, as per manufacturer's recommendation.

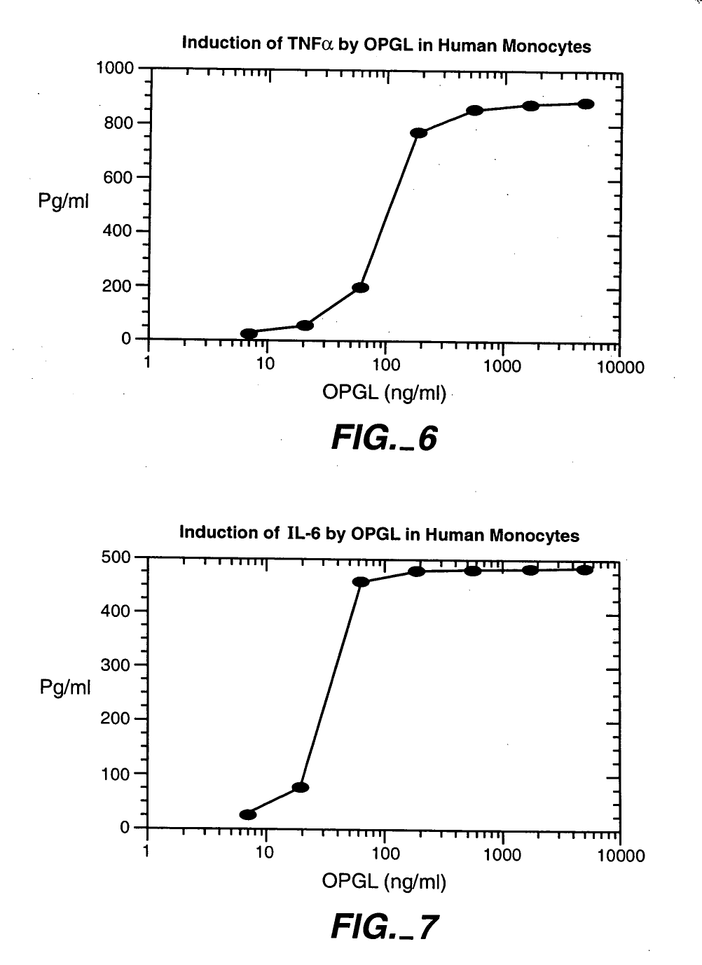

The results are shown in FIG. 6, and indicate the levels of TNF-alpha as Pg/ml.

### EXAMPLE 4

### Induction of IL-6 by OPG Ligand

An in vitro assay was conducted to examine the effects of OPG ligand on IL-6 induction in human monocytes. The assay was conducted essentially as described in Example 2. The supernatants were then measured by ELISA (Endogen) for IL-6 levels, as per manufacturer's recommendation.

The results are shown in FIG. 7, and indicate the levels of IL-6 as Pg/ml.

### EXAMPLE 5

### Induction of IL-1 by OPG Ligand

An in vitro assay was conducted to examine the effects of OPG ligand on IL-1 induction in human monocytes. The assay was conducted essentially as described in Example 2. The supernatants were then measured by ELISA (Endogen) for IL-1 levels, as per manufacturer's recommendation.

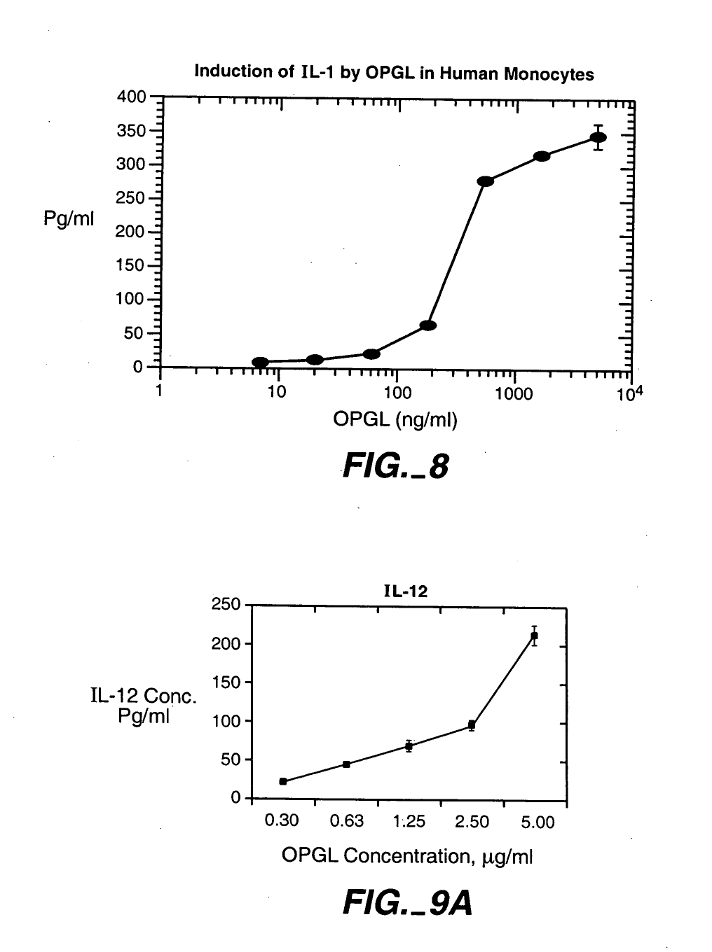

The results are shown in FIG. 8, and indicate the levels of IL-1 as Pg/ml.

### EXAMPLE 6

### Induction of Cytokine Secretion by OPG Ligand

In vitro assays were conducted to examine the effects of OPG ligand on secretion of various cytokines by human monocytes.

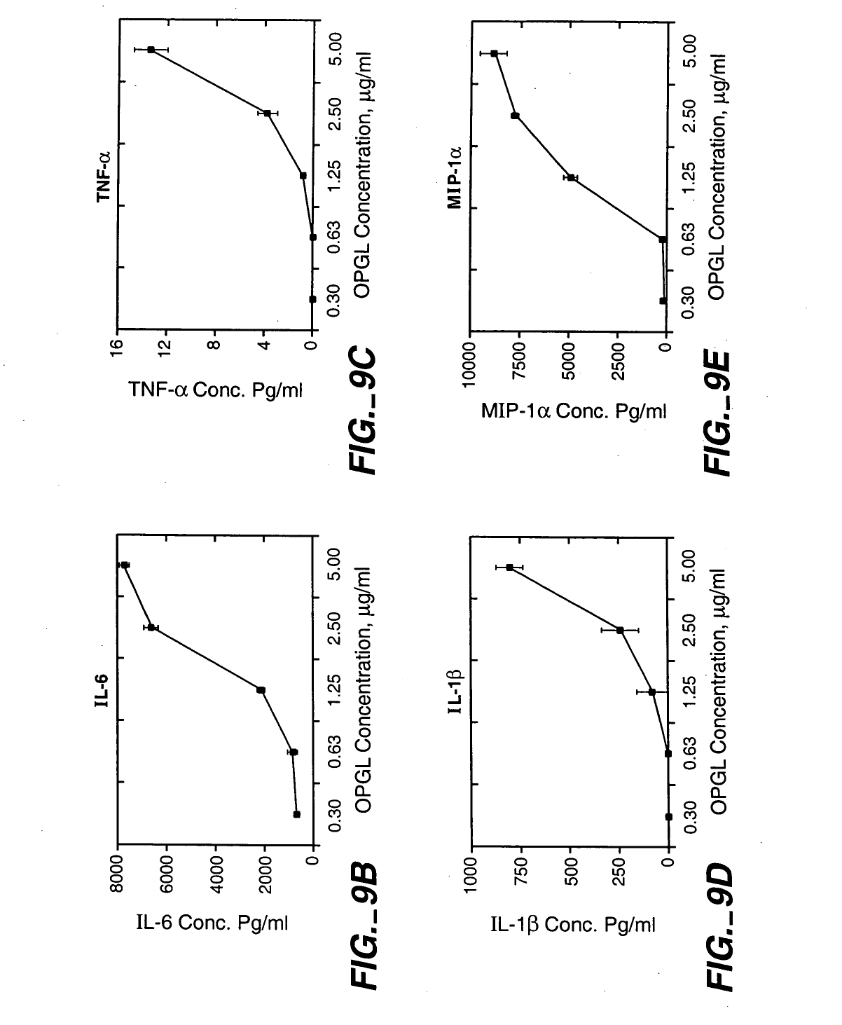

Monocytes were isolated from human peripheral blood using the Monocyte Isolation Kit (Milteny Biotec, cat # 553-01) as per manufacturer's recommendations. The cells were then resuspended in complete medium (RPMI-1640 containing 10% FBS heat-inactivated and 50 U/ml penicillin, 50 μg/ml streptomycin) and cultured at 37° C. for 24 hours with the indicated concentrations of OPG ligand (Alexis Corp.). The cell cultures were then tested for the cytokines (FIG. 9A-9E) by ELISA. ELISA kits obtained from Pharmingen were used to detect IL-12 and IL-6 levels and ELISA kits from R & D Systems were used to detect TNF-α, MIP-1α and IL-1β levels.

The results are shown in FIG. 9A-9E, and indicate the levels of IL-12, IL-6, TNF-α, MIP-1α and IL-1β secreted in pg/ml. The graphs clearly show activation of monocytes by OPGL in a dose-dependent manner, as evidenced by levels of IL-12 (213 pg/ml), IL-6 (7704 pg/ml), TNF-α (13.4 pg/ml), MIP-1α (8740 pg/ml) and IL-1β (803.8 pg/ml) at a maximal concentration of 5 μg/ml OPGL used.

### EXAMPLE 7

### OPG Ligand Induces Up-regulation of Co-stimulatory Molecule Expression on Monocytes

In vitro assays were conducted to examine the effects of OPG ligand on co-stimulatory molecule expression on monocytes.

Monocytes were isolated from human peripheral blood using the Monocyte Isolation Kit (Milteny Biotec, cat # 553-01) as per manufacturer's recommendations. The cells were then resuspended in complete medium (RPMI-1640 containing 10% FBS heat-inactivated and 50 U/ml penicillin, 50 μg/ml streptomycin) and cultured at 37° C. for 24 hours with or without 5 μg/ml OPG ligand (purchased from Alexis Corp.). Cells in the respective cultures at 0 and 24 hours were harvested gently using a cell scraper, washed with phosphate buffered saline containing 2% FBS heat inactivated, and adjusted to 3×10⁶ cells/ml in the same buffer. The cells were then incubated with either of the following antibodies for 15 minutes at 4° C. : phycoerythrin-conjugated α-human CD80 (Pharmingen), FITC-conjugated α-human CD86 (Pharmingen), phycoerythrin-conjugated α-human Class II (Pharmingen) or α-human RANK (Alexis Corp., cat # 804-212-C100). Cells stained with α-human RANK were washed with phosphate-buffered saline containing 2% FBS heat inactivated and were then incubated with FITC-conjugated α-mouse IgG1 antibody for 15 minutes at 4° C. Following incubation with the respective antibodies, cells were washed with phosphate-buffered saline containing 2% FBS heat inactivated and analyzed by FACS for expression of the co-stimulatory molecules CD80, CD86, and Class II as well as RANK.

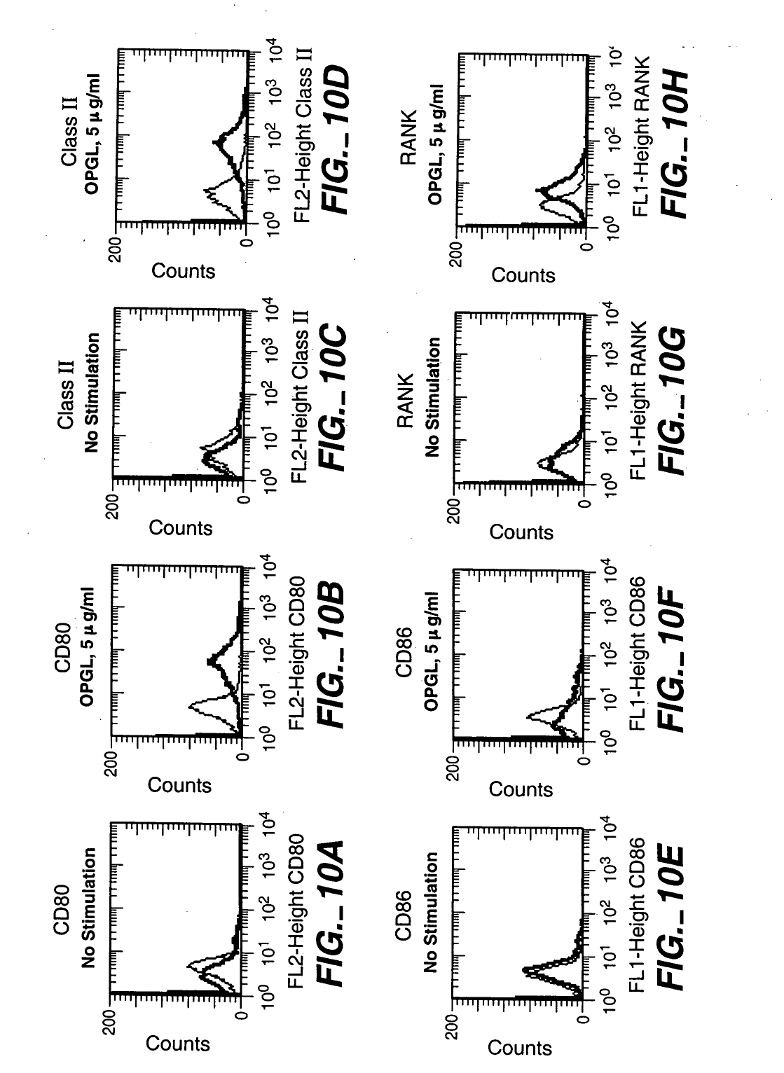

The results are shown in FIG. 10, wherein monocytes at 0 hours and 24 hours are illustrated in grey and bold lines respectively. FACS analyses of monocytes activated by OPGL (5 μg/ml) for 24 hours indicate up-regulation of activation markers such as CD80, CD86, and Class II, as well as RANK.

### EXAMPLE 8

### OPG Ligand Induces Proliferation of B-cells

In vitro assays were conducted to examine the effects of OPG ligand on human B cells.

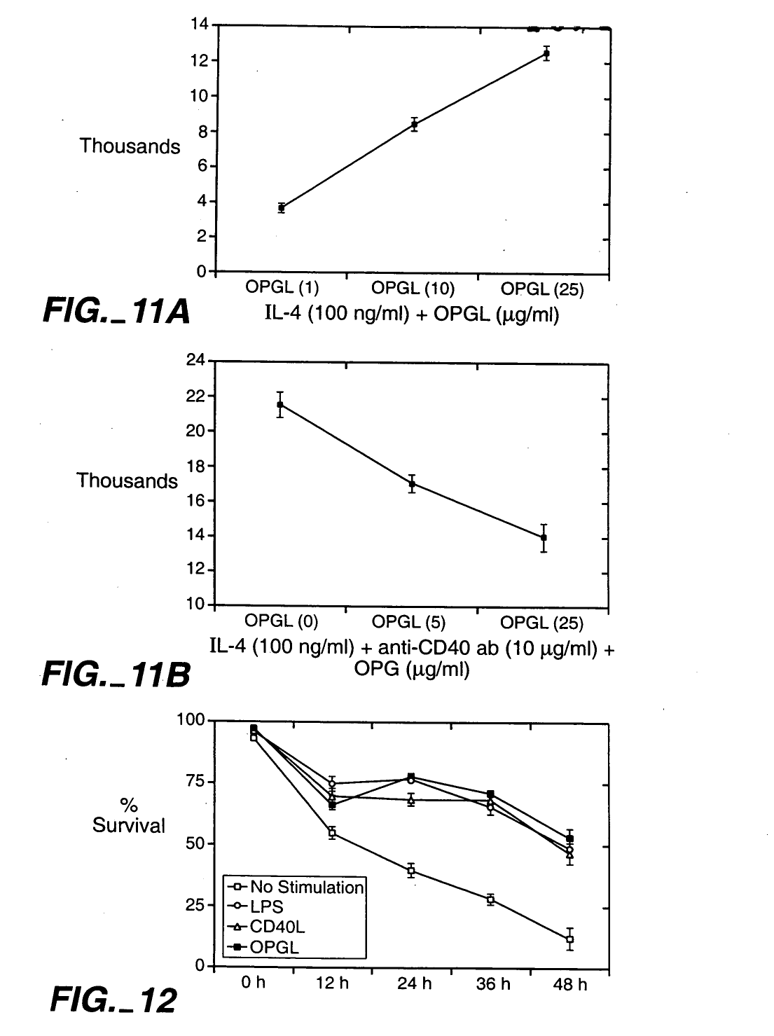

B cells were isolated from human peripheral blood using CD19 microbeads (Milteny Biotec, cat # 522-01) as per manufacturer's recommendations. Enriched B cells were resuspended in complete medium (RPMI-1640 containing 10% FBS heat-inactivated and 50 U/ml penicillin, 50 μg/ml streptomycin) and plated at 1×10⁶ cells/well in 96-well flat-bottom tissue culture plates. The cells were then cultured at 37° C. for 96 hours with 100 ng/ml rhuman IL-4 (R & D Systems, cat # 204-IL-025) and the indicated concentrations (see FIG. 11) of OPG ligand (Alexis Corp.). Proliferation of B cells was measured by pulsing the cultures with methyl 3H-thymidine (1 μCi/well) for an additional 16 hours. Thymidine incorporation was measured by scintillation counting.

The results are shown in FIG. 11A, and the proliferation of cells is reported as CPM×10⁻³. OPGL (in combination with IL-4 (100 ng/ml)) is thus able to induce proliferation of B cells in a dose-dependent manner.

In addition, the effects of OPG in attenuating α-CD40 antibody induced proliferation of B cells were examined. The assays were conducted essentially as described above, except that the cells were incubated with 100 ng/ml rhuman IL-4 (R & D Systems, cat # 204-IL-025), 10 μg/ml α-human CD40 antibody (Pharmingen, cat # 33070D), and the indicated concentrations (see FIG. 11B) of OPG (Alexis Corp.).

The results are shown in FIG. 11B, and the proliferation of cells is reported as CPM×10⁻³. OPG is thus able to block proliferation of B cells mediated by α-human CD40 antibody in combination with IL-4, in a dose-dependent manner.

### EXAMPLE 9

### OPG Ligand Protects Monocytes from Apoptosis Induced by Serum-Starvation

In vitro assays were conducted to examine the effects of OPG ligand on human monocytes in serum-starved cultures.

Monocytes were isolated from human peripheral blood using the Monocyte Isolation Kit (Milteny Biotec, cat # 553-01) as per manufacturer's recommendations. The cells were then resuspended in serum-free medium (RPMI-1640 containing 50 U/ml penicillin, 50 μg/ml streptomycin) at 5×10⁵ cells/ml, and cultured at 37° C. for the time period of hours indicated in FIG. 12 in the presence of 0.5 mg/ml LPS (Sigma, Cat # L-4391), 1 μg/ml CD40 ligand (Alexis Corp.), or 1 μg/ml OPG ligand (Alexis Corp.). At the indicated time points (see FIG. 12), cells in the respective cultures were stained with Annexin V-FITC (Clontech Laboratories, cat # K2025-2) and analyzed by FACS as per manufacturer's instructions.

The results are shown in FIG. 12. They clearly indicate the ability of OPGL to protect monocytes from apoptosis induced by serum-withdrawal, and this anti-apoptotic ability of OPGL is comparable to that of known survival stimuli such as LPS and CD40L.

### EXAMPLE 10

### OPG Ligand Induces Expression of Bcl-x1 and Bcl-2 in Monocytes

In vitro assays were conducted to examine the effects of OPG ligand on expression of certain survival proteins in human monocytes.

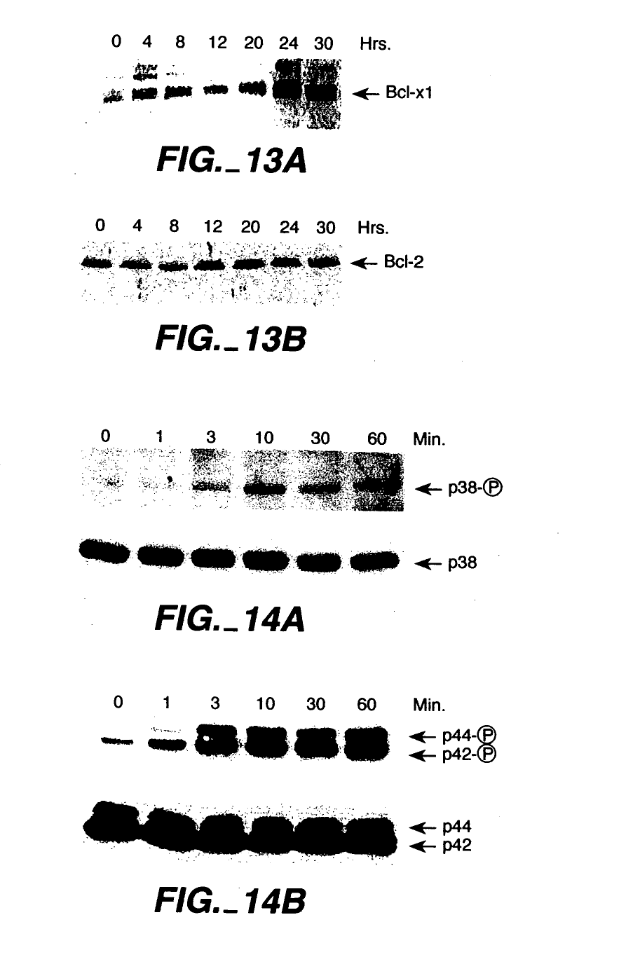

Monocytes were isolated from human peripheral blood using the Monocyte Isolation Kit (Milteny Biotec, cat # 553-01) as per manufacturer's recommendations. The cells were then resuspended in complete medium (RPMI-1640 containing 10% FBS heat-inactivated and 50 U/ml penicillin, 50 μg/ml streptomycin) at 5.×10⁵ cells/ml and cultured at 37° C. with 1 μg/ml OPG ligand (Alexis Corp.). At the indicated time points (see FIG. 13), cells were harvested, washed once with phosphate-buffered saline, and lysed in buffer (1% SDS, 0.5% Nonidet P-40, 0.15 M NaCl, 10 mM Tris (pH 7.4), and 1 tablet complete protease inhibitor mixture (Roche Molecular Biochemicals). The lysates were centrifuged at 10,000×g for 15 minutes at 4° C. The supernatant was collected and used as lysate. Lysates (30 or 50 μg) were separated via SDS-polyacrylamide gel electrophoresis using 4-20% Tris-glycine gels (Novex Electrophoresis) in SDS Running buffer (25 mM TRIS, 0.2 M glycine and 3.5 mM SDS), and transferred onto polyvinylidene difluoride membrane (Invitrogen Corp.) in transfer buffer (48 mM Tris-Base, 39 mM Glycine, 0.0375%(w/v) SDS, 20% Methanol). The membrane was incubated in blocking buffer composed of 5% skim milk in TBST (20 mM Tris (pH 7.4), 137 mM NaCl, 0.5% Tween 20) followed by primary antibodies for Bcl-2 (Pharmingen cat # 554202) or Bcl-xL ((Pharmingen cat # 556499). Antibody-antigen complexes were detected using a horseradish peroxidase-conjugated secondary antibody and ECL system (Amersham Pharmacia Biotech).

The results are shown in FIG. 13. Thus OPGL's ability to block apoptosis induced by serum-withdrawal in monocytes (see FIG. 12) may be mediated by induction of pro-survival protein expression such as Bcl-xL and Bcl-2.

### EXAMPLE 11

### OPG Ligand Induces Activation of MAPK p38 and p42/44 Pathways in Monocytes

In vitro assays were conducted to examine the effects of OPG ligand on expression of certain survival proteins in human monocytes.

Monocytes were isolated from human peripheral blood using the Monocyte Isolation Kit (Milteny Biotec, cat # 553-01) as per manufacturer's recommendations, and serum-starved in serum-free medium (RPMI-1640 containing 50 U/ml penicillin,50 μg/ml streptomycin) for 6 hours at 37° C. The cells were then harvested gently using a cell scraper, resuspended in complete medium (RPMI-1640 containing 10% FBS heat-inactivated and 50 U/ml penicillin, 50 μg/ml streptomycin) at 1×10⁶ cells/ml, and stimulated with 1 μg/ml OPG ligand (Alexis Corp.). At the indicated time points (see FIG. 14), cells were harvested, washed once with phosphate-buffered saline, and lysed in buffer (20 mM Hepes, pH 7.4, 2 mM EGTA, 50 mM -glycerophosphate, 0.1% Triton X-100, 10% glycerol, 1 mM dithiothreitol, 1 tablet complete protease inhibitor mixture (Roche Molecular Biochemicals)) The lysates were centrifuged at 10,000×g for 15 minutes at 4° C. The supernatant was collected and used as whole cell lysate. Lysates (30 or 50 μg) were separated via SDS-polyacrylamide gel electrophoresis using 4-20% Tris-glycine gels (Novex Electrophoresis) in SDS Running buffer (25 mM Tris, 0.2 M glycine and 3.5 mM SDS), and transferred onto polyvinylidene difluoride membrane (Invitrogen Corp.) in transfer buffer (48 mM Tris-Base, 39 mM Glycine, 0.0375% (w/v) SDS, 20% Methanol). The membrane was incubated in blocking buffer composed of 5% skim milk in TBST (20 mM Tris (pH 7.4), 137 mM NaCl, 0.5% Tween 20) followed by primary antibodies for p38 MAPK (Cell Signaling Technology), phospho-p38 MAPK (Cell Signaling Technology), p42/44 MAPK (Cell Signaling Technology) or phospho-p42/44 MAPK (Cell Signaling Technology). Antibody-antigen complexes were detected using a horseradish peroxidase-conjugated secondary antibody and ECL system (Amersham Pharmacia Biotech).

The results are shown in FIG. 14, and demonstrate activation of p38 and p42/44 MAPK pathways in monocytes by OPGL.

### EXAMPLE 12

OPG Ligand and RANK Expression in Normal and Diseased Cells or Tissues

Assays were conducted to examine the expression of OPG ligand and RANK in various cells and tissues.

Monocytes were isolated from human peripheral blood using the Monocyte Isolation Kit (Milteny Biotec, cat # 553-01) as per manufacturer's recommendations. The cells were resuspended in phosphate buffered saline containing 2% FBS heat inactivated, and adjusted to 1×10⁶ cells/ml. The cells were then incubated with the α-human RANK (Alexis Corp., cat # 804-212-C100) or isotype control antibody (Pharmingen) for 15 minutes at 4° C. Cells from respective incubations were washed with phosphate-buffered saline containing 2% FBS heat inactivated and then incubated with FITC-conjugated α-mouse IgG1 antibody for 15 minutes at 4° C. Following this incubation, cells were washed with phosphate-buffered saline containing 2% FBS heat inactivated and analyzed by FACS for expression of RANK.

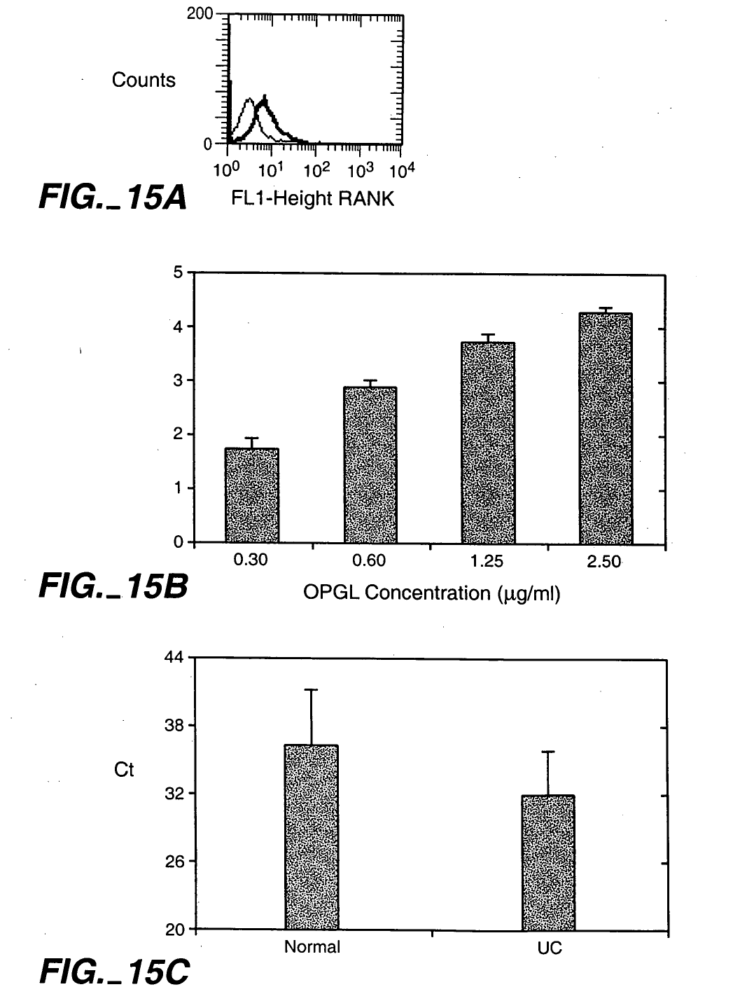

The results are shown in FIG. 15A, wherein the RANK-stained cells are illustrated in a bold line and the isotype-control-stained cells are illustrated in grey. Thus, RANK, the membrane-bound receptor for OPGL, is expressed on resting monocytes.

RANK mRNA expression was found to be upregulated in monocytes treated with OPG ligand. Monocytes were isolated from human peripheral blood using the Monocyte Isolation Kit (Milteny Biotec, cat # 553-01) as per manufacturer's recommendations. The cells were then resuspended in complete medium (RPMI1640 containing 10% FBS heat-inactivated and 50 U/ml penicillin, 50 ug/ml streptomycin) at 1×10⁶ cells/ml and cultured at 37° C. for 24 hours with (or without) the indicated concentrations of OPG ligand (Alexis Corporation) (see FIG. 15B). Total RNA was then isolated from OPGL-treated and control cells using TRIzol™ reagent (Life Technologies) as per manufacturer's recommendations. Taqman amplification reactions (50 μl) consisted of 25 ng of RNA sample and 40 ul of a reaction cocktail. The reaction cocktail contained 10× buffer A, 10 Units RNase inhibitor, 200 uM DATP, dCTP, dGTP, dTTP, 4 mM MgCl₂, 1.25 Units Taq Gold™ Polymerase and 25 Units MULV reverse transcriptase (Taqman Core Kit (Perkin Elmer, cat # N808-0228). Each well contained a 10 μl primer/probe mix of 200 nM gene-specific hybridization probe, and 300 nM gene-specific amplification primers.

Thermal cycling conditions: 30 minutes at 48° C., then 2 minutes at 50° C. and 10 minutes at 95° C. The reactions then cycled 40 times with 15 seconds at 95° C. and 1 minute at 60° C. Reactions and sequence detection were conducted with the ABI Prism 7700 Sequence Detector. GAPDH levels were used to normalize loading.

The sequences of the RANK/GAPDH Taqman primer/probe set used are as follows:
RANK Forward primer: 5′-AGTGGTGCGATTATAGCCCG-3′ (SEQ ID NO:7)
RANK Reverse primer: 5′-GAAGGTTGAGGTGGGAGGATC-3′ (SEQ ID NO:8)
RANK Probe: 5′-AGCCTCTAACTCCTGGGCTCAAGCAATC-3′ (SEQ ID NO:9)
GAPDH Forward primer: 5′-TGGGCTACACTGAGCACCAG-3′ (SEQ ID NO:10)
GAPDH Reverse primer: 5′-CAGCGTCAAAGGTGGAGGAG-3′ (SEQ ID NO:11)
GAPDH Probe: 5′-TGGTCTCCTCTGACTTCAACAGCGACAC-3′ (SEQ ID NO:12)

Fold-increase in RANK transcript expression of OPGL-treated cells over unstimulated cells is shown in FIG. 15B. OPGL is thus able to stimulate RANK mRNA expression in monocytes in a dose-dependent manner.

OPG ligand mRNA expression was found to be up-regulated in colon tissues of ulcerative colitis patients. Colon tissues from normal, healthy donors and from ulcerative colitis patients were obtained. Total RNA was isolated from the tissues by Caesium Chloride gradient centrifugation. Amplification reactions (50 ul) consisted of 25 ng of RNA sample and 40 ul of a reaction cocktail. The reaction cocktail contained 10× buffer A, 10 Units RNase inhibitor, 200 uM DATP, dCTP, dGTP, dTTP, 4 mM MgCl₂, 1.25 Units Taq Gold™ Polymerase and 25 Units MULV reverse transcriptase (Taqman Core Kit (Perkin Elmer, cat # N808-0228). Each well contained a 10 ul primer/probe mix of 200 nM gene-specific hybridization probe, and 300 nM gene-specific amplification primers.

Thermal cycling conditions: 30 minutes at 48° C, then 2 minutes at 50° C. and 10 minutes at 95° C. The reactions then cycled 40 times with 15 seconds at 95° C. and 1 minute at 60° C. Reactions and sequence detection were conducted with the ABI Prism 7700 Sequence Detector.

The sequences of the OPGL/GAPDH Taqman primer/probe set used are as follows:
OPGL Forward primer: 5′-CAAGTATTGGTCAGGGAATTCTG-3′ (SEQ ID NO:13)
OPGL Reverse primer: 5′-GGGCTCAATCTATATCTCGAACTT-3′ (SEQ ID NO:14)
OPGL Probe: 5′-FAM-TTTAAGTTACGGTCTGGAGAGGAAATCAGCA-TAMARA-3′ (SEQ ID NO:15)
GAPDH Forward primer: 5′-GAAGGTGAAGGTCGGAGTC-3′ (SEQ ID NO:16)
GAPDH Reverse primer: 5′-GAAGATGGTGATGGGATTTC-3′ (SEQ ID NO:17)
GAPDH Probe: 5′-FAM-CAAGCTTCCCGTTCTCAGCC-TAMARA-3′ (SEQ ID NO:18)

Taqman Cₜ values for OPGL mRNA expression in normal and ulcerative colitis tissues are shown in FIG. 15C. The results indicate that levels of OPGL mRNA may be upregulated at least 8-fold in ulcerative colitis tissues over normal tissues, suggesting that OPGL may play a role in the pathogenesis of the disease.

The foregoing written description is considered to be sufficient to enable one skilled in the art to practice the invention. The present invention is not to be limited in scope by the example presented herein. Indeed, various modifications of the invention in addition to those shown and described herein will become apparent to those skilled in the art from the foregoing description and fall within the scope of the appended claims.

1. A method of stimulating mammalian monocytes, comprising exposing said mammalian monocytes to an effective amount of OPG ligand polypeptide that stimulates said mammalian monocytes to secrete one or more cytokines or chemokines selected from the group consisting of IL-1, IL-6, TNF-alpha, and IL-8, wherein said OPG ligand polypeptide comprises:
a) a polypeptide having at least 80% sequence identity to the full length native sequence OPG ligand polypeptide having the amino acid sequence of FIG. 1B (SEQ ID NO:1);
b) a soluble, extracellular domain sequence of the polypeptide of FIG. 1B (SEQ ID NO:1);
c) a polypeptide consisting of the amino acid sequence of FIG. 1B (SEQ ID NO:1); or
d) a polypeptide comprising a fragment of a), b) or c).

2. The method of claim 1 wherein said mammalian monocytes are exposed to said OPG ligand polypeptide in vitro.

3. The method of claim 1 wherein said mammalian monocytes are exposed to said OPG ligand polypeptide in vivo.

4. The method of claim 1 wherein said OPG ligand polypeptide stimulates said mammalian monocytes to secrete IL-1.

5. The method of claim 4 wherein said IL-1 is IL-1β.

6. The method of claim 1 wherein said OPG ligand polypeptide stimulates said mammalian monocytes to secrete IL-6.

7. The method of claim 1 wherein said OPG ligand polypeptide stimulates said mammalian monocytes to secrete TNF-alpha.

8. The method of claim 1 wherein said OPG ligand polypeptide stimulates said mammalian monocytes to secrete IL-8.

9. The method of claim 1 wherein said OPG ligand polypeptide comprises a soluble, extracellular domain sequence of the polypeptide of FIG. 1B (SEQ ID NO:1).

10. The method of claim 9 wherein said OPG ligand polypeptide extracellular domain comprises amino acids 70 to 317 of FIG. 1B (SEQ ID NO:1).

11. The method of claim 1 wherein said OPG ligand polypeptide has at least 80% sequence identity to the full length native sequence OPG ligand polypeptide having the amino acid sequence of FIG. 1B (SEQ ID NO:1).

12. The method of claim 11 wherein said OPG ligand polypeptide has at least 90% sequence identity.

13. A method of stimulating mammalian monocytes, comprising exposing said mammalian monocytes to an effective amount of OPG ligand polypeptide that stimulates said mammalian monocytes to secrete one or more cytokines or chemokines selected from the group consisting of IL-12 and MIP-1α, wherein said OPG ligand polypeptide comprises:
a) a polypeptide having at least 80% sequence identity to the full length native sequence OPG ligand polypeptide having the amino acid sequence of FIG. 1B (SEQ ID NO:1);
b) a soluble, extracellular domain sequence of the polypeptide of FIG. 1B (SEQ ID NO:1);
c) a polypeptide consisting of the amino acid sequence of FIG. 1B (SEQ ID NO:1); or
d) a polypeptide comprising a fragment of a), b) or c).

14. The method of claim 13 wherein said mammalian monocytes are exposed to said OPG ligand polypeptide in vitro.

15. The method of claim 13 wherein said mammalian monocytes are exposed to said OPG ligand polypeptide in vivo.

16. The method of claim 13 wherein said OPG ligand polypeptide stimulates said mammalian monocytes to secrete IL-12.

17. The method of claim 13 wherein said OPG ligand polypeptide stimulates said mammalian monocytes to secrete MIP-1α.

18. The method of claim 13 wherein said OPG ligand polypeptide comprises a soluble, extracellular domain sequence of the polypeptide of FIG. 1B (SEQ ID NO:1).

19. The method of claim 13 wherein said OPG ligand polypeptide has at least 80% sequence identity to the full length native sequence OPG ligand polypeptide having the amino acid sequence of FIG. 1B (SEQ ID NO:1).

20. The method of claim 9 wherein said OPG ligand polypeptide has at least 90% sequence identity.

21. A method of stimulating mammalian monocytes, comprising exposing said mammalian monocytes to an effective amount of agonist anti-RANK receptor antibody that stimulates said mammalian monocytes to secrete one or more cytokines or chemokines selected from the group consisting of IL-1, IL-6, IL-12, TNF-alpha, MIP-1α, and IL-8.

22. The method of claim 21 wherein said mammalian monocytes are exposed to said agonist anti-RANK receptor antibody in vitro.

23. The method of claim 21 wherein said mammalian monocytes are exposed to said agonist anti-RANK receptor antibody in vivo.

24. The method of claim 21 wherein said agonist anti-RANK receptor antibody stimulates said mammalian monocytes to secrete IL-1.

25. The method of claim 21 wherein said agonist anti-RANK receptor antibody stimulates said mammalian monocytes to secrete IL-6.

26. The method of claim 21 wherein said agonist anti-RANK receptor antibody stimulates said mammalian monocytes to secrete IL-12.

27. The method of claim 21 wherein said agonist anti-RANK receptor antibody stimulates said mammalian monocytes to secrete MIP-1α.

28. The method of claim 21 wherein said agonist anti-RANK receptor antibody stimulates said mammalian monocytes to secrete TNF-alpha.

29. The method of claim 21 wherein said agonist anti-RANK receptor antibody stimulates said mammalian monocytes to secrete IL-8.

30. The method of claim 21 wherein said agonist anti-RANK receptor antibody is a monoclonal antibody.

31. The method of claim 30 wherein said agonist anti-RANK receptor antibody is a chimeric, humanized or human antibody.

32. A method of inhibiting mammalian monocytes, comprising exposing said mammalian monocytes to an effective amount of antagonist that inhibits secretion of one or more cytokines or chemokines by said mammalian monocytes, wherein said antagonist comprises an anti-OPG ligand antibody, an anti-OPG receptor antibody, an anti-RANK receptor antibody, an OPG receptor immunoadhesin or a RANK receptor immunoadhesin, and said one or more cytokines or chemokines are selected from the group consisting of IL-1, IL-6, IL-12, MIP-1α, TNF-alpha, and IL-8.

33. The method of claim 32 wherein said mammalian monocytes are exposed to said antagonist in vitro.

34. The method of claim 32 wherein said mammalian monocytes are exposed to said antagonist in vivo.

35. The method of claim 32 wherein said antagonist inhibits secretion of IL-1 by said mammalian monocytes.

36. The method of claim 32 wherein said antagonist inhibits secretion of IL-6 by said mammalian monocytes.

37. The method of claim 32 wherein said antagonist inhibits secretion of IL-12 by said mammalian monocytes.

38. The method of claim 32 wherein said antagonist inhibits secretion of MIP-1α by said mammalian monocytes.

39. The method of claim 32 wherein said antagonist inhibits secretion of TNF-alpha by said mammalian monocytes.

40. The method of claim 32 wherein said antagonist inhibits secretion of IL-8 by said mammalian monocytes.

41. The method of claim 32 wherein said antagonist is an anti-RANK receptor antibody.

42. The method of claim 41 wherein said anti-RANK receptor antibody is a chimeric, humanized or human antibody.

43. The method of claim 32 wherein said antagonist is a RANK receptor immunoadhesin.

44. The method of claim 43 wherein said RANK receptor immunoadhesin comprises an extracellular domain of the RANK receptor and an immunoglobulin constant domain.

45. The method of claim 44 wherein said extracellular domain of the RANK receptor comprises amino acids 29 to 212 of FIG. 3B (SEQ ID NO:5) or a fragment thereof.

46. A method of treating a pathological condition associated with or resulting from decreased cytokine or chemokine secretion by mammalian monocytes, comprising administering to a mammal an effective amount of agonist to stimulate the mammal's monocytes to secrete one or more cytokines or chemokines selected from the group consisting of IL-1, IL-6, IL-12, MIP-1α, TNF-alpha, and IL-8, wherein the agonist comprises:
a) a polypeptide having at least 80% sequence identity to the full length native sequence OPG ligand polypeptide having the amino acid sequence of FIG. 1B (SEQ ID NO:1);
b) a soluble, extracellular domain sequence of the polypeptide of FIG. 1B (SEQ ID NO:1);
c) a polypeptide consisting of the amino acid sequence of FIG. 1B (SEQ ID NO:1);
d) a polypeptide comprising a fragment of a), b) or c); or
e) an anti-RANK receptor antibody.

47. The method of claim 46 wherein said pathological condition is an immune related condition.

48. The method of claim 47 wherein said immune related condition is an infectious disease.

49. The method of claim 46 wherein said anti-RANK receptor antibody is a monoclonal antibody.

50. The method of claim 49 wherein said antibody is a chimeric, humanized or human antibody.

51. A method of treating a pathological condition associated with or resulting from increased cytokine or chemokine secretion by mammalian monocytes, comprising administering to a mammal an effective amount of antagonist to inhibit secretion of one or more cytokines or chemokines selected from the group consisting of IL-1, IL-6, IL-12, MIP-1α, TNF-alpha, and IL-8 by said mammal's monocytes, wherein the antagonist comprises an anti-OPG ligand antibody, an anti-OPG receptor antibody, an anti-RANK receptor antibody, an OPG receptor immunoadhesin or a RANK receptor immunoadhesin.

52. The method of claim 51 wherein said pathological condition is an immune related condition.

53. The method of claim 52 wherein said immune related condition is autoimmune disease, rheumatoid arthritis, insulin dependent diabetes, osteoarthritis, inflammatory bowel disease, psoriasis, transplant rejection or allergy.

54. The method of claim 53 wherein said immune related condition is rheumatoid arthritis.

55. The method of claim 53 wherein said inflammatory bowel disease is ulcerative colitis or Crohn's disease.

56. The method of claim 51 wherein said anti-OPG ligand antibody, anti-OPG receptor antibody, or anti-RANK receptor antibody is a monoclonal antibody.

57. The method of claim 56 wherein said monoclonal antibody is a chimeric, humanized or human antibody.

58. The method of claim 51 wherein said antagonist is a RANK receptor immunoadhesin or OPG receptor immunoadhesin.

59. The method of claim 58 wherein said RANK receptor immunoadhesin comprises an extracellular domain of the RANK receptor and an immunoglobulin constant domain.

60. The method of claim 59 wherein said extracellular domain of the RANK receptor comprises amino acids 29 to 212 of FIG. 3B (SEQ ID NO:5) or a fragment thereof.

61. A method of treating rheumatoid arthritis or inflammatory bowel disease in a mammal, comprising administering to the mammal an effective amount of antagonist to inhibit one or more cytokines or chemokines selected from the group consisting of IL-1, IL-6, IL-12, MIP-1α, TNF-alpha, and IL-8, wherein the antagonist comprises an anti-OPG ligand antibody, an anti-OPG receptor antibody, an anti-RANK receptor antibody, an OPG receptor immunoadhesin or a RANK receptor immunoadhesin.

62. The method of claim 61 wherein said antagonist is a RANK receptor immunoadhesin or OPG receptor immunoadhesin.

63. The method of claim 62 wherein said RANK receptor immunoadhesin comprises an extracellular domain of the RANK receptor and an immunoglobulin constant domain.

64. The method of claim 63 wherein said extracellular domain of the RANK receptor comprises amino acids 29 to 212 of FIG. 3B (SEQ ID NO:5) or a fragment thereof.

65. An article of manufacture, comprising:
(a) a composition of matter comprising an effective amount of the OPG ligand polypeptide of claim 1 or 13, the agonist of claim 21, or antagonist of claim 32 or 46;
(b) a container containing said composition; and (c) a label affixed to said container, or a package insert included in said container referring to the use of said OPG ligand polypeptide or agonist or antagonist in the treatment of an immune related disease.
